(19) United States
(12) Patent Application Publication
(10) Pub. No.: US 2024/0410019 A1
(43) Pub. Date: Dec. 12, 2024

(54) CLUSTERED MUTATIONS FOR THE TREATMENT OF CANCER

(71) Applicant: The Regents of the University of California, Oakland, CA (US)

(72) Inventors: Ludmil B. Alexandrov, San Diego, CA (US); Erik N. Bergstrom, La Jolla, CA (US)

(21) Appl. No.: 18/719,020

(22) PCT Filed: Dec. 13, 2022
(86) PCT No.: PCT/US2022/052745

§ 371 Date: Jun. 12, 2024

Related U.S. Application Data
(60) Provisional application No. 63/289,601, filed on Dec. 14, 2021.

(51) Int. Cl. C12Q 1/6886

(52) U.S. Cl. CPC C12Q 1/6886; C12Q 2600/106; C12Q 2600/156

(57) ABSTRACT

The genomes of cancer cells harbor somatic mutations imprinted by the activities of different mutational processes. Most single-base substitutions and small insertions and deletions (indels) are independently scattered across the genomic landscape; however, a subset o substitutions and indels tend to cluster together. This clustering has been attributed to a combination of heterogeneous mutation rates across the genomic landscape, biophysical characteristics of exogenous carcinogens, dysregulation of endogenous processes, and the occurrence of larger events associated with genome instability; amongst others. Diagnostic and therapeutic methods are disclosed herein that utilize a clustered mutation in one or more genes in a sample isolated from a subject.

### CROSS-REFERENCE TO RELATED APPLICATION

This application claims priority under 35 U.S.C. § 119 (e) to U.S. Provisional Application No. 63/289,601, filed Dec. 14, 2021, the contents of which are incorporated herein by reference in their entireties.

### BACKGROUND

Throughout this disclosure, reference is made to technical and patent literature. In some aspects, the literature is referenced by an Arabic numeral, the complete bibliographic citation for which immediately preceding the claims. These disclosures are provided to describe the state of the art to which this disclosure pertains and are incorporated herein by reference.

The genomes of cancer cells harbor somatic mutations imprinted by the activities of different mutational processes^{1,2}. Most single-base substitutions and small insertions and deletions (indels) are independently scattered across the genomic landscape; however, a subset of substitutions and indels tend to cluster together^{3,4}. This clustering has been attributed to a combination of heterogeneous mutation rates across the genomic landscape, biophysical characteristics of exogenous carcinogens, dysregulation of endogenous processes, and the occurrence of larger events associated with genome instability; amongst others⁴⁻¹⁶. Prior analyses of clustered mutations have focused on single-base substitutions and revealed several classes of clustered events, including doublet-and multi-base substitutions^{1,9,16-19}, diffuse hypermutation termed omikli¹⁵, and longer events termed kataegis^{12,14,16,20}. The majority of kataegic events were found to be strand-coordinated which is usually defined as sharing the same strand and reference allele^{2,16}. Previous studies have also revealed 9 clustered mutational signatures⁴ of different mutational processes as well as clustered driver substitutions due to APOBEC3-associated mutagenesis¹⁵ or due to carcinogenic-triggered POLH mutagenesis⁴. To the best of Applicant's knowledge, analysis of cluster indels or a comprehensive exploration of clustered driver mutations has never been performed.

### SUMMARY OF THE DISCLOSURE

Doublet-base substitutions have been extensively examined revealing multiple endogenous and exogenous mutational processes that can cause these events, including, failure of DNA repair pathways and exposure to environmental mutagens^{1,2,16,17}. In contrast, multi-base substitutions have not been comprehensively explored presumably due to their small numbers in most cancer genomes. Moreover, only a handful of reported processes have been associated with omikli and kataegic events with majority of these processes attributed to AID/APOBEC3 family of deaminases^{4,5,12,14-16,21-24}. For example, in B-cell lymphomas clustered tracks of C>T and C>G mutations at WRCY motifs are the result of direct replication over AID lesions²¹. Alternatively, AID-induced lesions can be processed by the mismatch repair pathway that recruits the error-prone DNA polymerase n resulting in non-canonical AID mutations²¹. In addition to AID, the APOBEC3 enzymes, which are typically responsible for anti-viral responses and for limiting the mobility of mobile elements²⁵⁻³¹, are a substantial contributor of clustered mutational events^{2,4,12,14-16,24,32}. Specifically, the APOBEC3 enzymes give rise to omikli and kataegis by requiring single-stranded DNA as a substrate^{14,15,24,32}. Omikli were found enriched in early replicating regions and more prevalent in microsatellite stable tumors indicating a role of mismatch repair in exposing short single-stranded DNA regions while processing mismatched bases during replication¹⁵. Further, the differential activity of mismatch repair towards gene-rich regions results in an increased mutational burden of omikli mutations within cancer driver genes¹⁵. Kataegis is less prevalent than omikli as it likely depends on longer tracks of single-stranded DNA¹²⁻¹⁴ Such tracks are typically available during repair of double-strand breaks and the majority of kataegis has been observed within 10 kb of detected breakpoints¹¹.

Amplification of known cancer genes due to double-strand breaks and complex rearrangements is known to drive tumorigenesis in many cancer types³³. Recent studies have elucidated high copy number states of circular extrachromosomal DNA (ecDNA), which often harbor known cancer genes and have been found in most human cancers³³⁻³⁶. The circular nature of ecDNAs and their rapid replication patterns mimic double stranded DNA viral pathogens indicating a potential substrate for APOBEC3 mutagenesis, which may ultimately contribute to the subclonal diversification of tumors harboring ecDNA through accelerated diversification of the extrachromosomal oncoproteins.

Described herein is a comprehensive examination of clustered substitutions and clustered indels across 2,583 cancer genomes spanning 30 different tumor types. The results elucidate a multitude of mutational processes giving rise to clustered mutations, including clustered driver mutations that associated with differential gene expression and changes in overall survival, and reveal recurrent APOBEC3 mutagenesis, termed kyklonas, fueling the evolution of ecDNA.

The application of these discoveries are further provided herein. In one aspect, a method of treating inhibiting the growth of a cancer cell or treating a cancer in a subject in need thereof, wherein the subject has one or more of TP53, EGFR, KIT, KMT2C, ELF3, APC and ARID1A or lacks a clustered mutation in a BRAF gene in a sample isolated from the subject is disclosed. The method comprises, consists of, or consists essentially of administering an aggressive therapy to the subject, thereby inhibiting the growth of the cancer cell or treating the cancer in the subject. If the subject does not have a clustered mutation in one or more of TP53, EGFR, KIT, KMT2C, ELF3, APC and ARID1A or lacks a clustered mutation in a BRAF gene, a less aggressive therapy can be administered to the therapy.

In yet another aspect, a method for selecting a cancer patient for an aggressive therapy is disclosed. The method comprises, consists of, or consists essentially of assaying for and/or detecting at least one clustered mutation in a gene selected from TP53, EGFR, KIT, KMT2C, ELF3, APC and ARID1A and/or no clustered mutation in a BRAF gene in a sample isolated from the subject wherein the subject is selected for the therapy if the one or more clustered mutations are found in TP53, EGFR, KIT, KMT2C, ELF3, APC and/or ARID1A and/or no BRAF gene clustered mutation is detected in the sample isolated from the cancer patient.

Cancer patients determined to have mutations that have better predictive outcomes, such as a longer overall survival will in one aspect can receive therapy, but a less aggressive can be selected for the initial or subsequent therapy.

In yet another aspect, a method for identifying whether a cancer patient is likely to experience a relatively longer or shorter overall survival is disclosed. The method comprises, consists of, or consists essentially of assaying for and/or detecting at least one clustered mutation in a gene selected from TP53, EGFR, KIT, KMT2C, ELF3, APC and ARID1A or a BRAF gene in a sample isolated from the patient, wherein the patient is likely to experience longer overall survival if the clustered mutation is detected in BRAF or a clustered mutation is not detected in the one clustered mutation in a gene selected from TP53, EGFR, KIT, KMT2C, ELF3, APC and ARID1A, and the patient is likely to experience shorter overall survival if the clustered mutation is detected in one clustered mutation in a gene selected from TP53, EGFR, KIT, KMT2C, ELF3, APC and ARID1A, or not detected in the BRAF gene.

### BRIEF DESCRIPTION OF THE DRAWING

FIGS. 1A-1B show the landscape of clustered mutations across human cancer.

FIG. 1A: Pan-cancer distribution of clustered substitutions subclassified into doublet-base substitutions (DBSs), multi-base substitutions (MBSs), omikli, kataegis, and other clustered mutations. Top panel: Each black dot represents a single cancer genome. Gray bars reflect the median clustered tumor mutational burden (TMB) for cancer types. Middle panel: The clustered TMB normalized to the genome wide TMB reflecting the contribution of clustered mutations to the overall TMB of a given sample. Gray bars reflect the median contribution for cancer types. Bottom panel: The proportion of each subclass of clustered events for a given cancer type with the total number of samples having at least a single clustered event over the total number of samples within a given cancer cohort. FIG. 1B: Pan-cancer distribution of clustered small insertions and deletions. Top and Middle panels have the same information as panel FIG. 1A. Bottom panel: The proportion of each cluster type of indel for a given cancer type with the total number of samples having at least a single clustered indel over the total number of samples within a given cancer cohort. All 2,583 whole-genome sequenced samples from PCAWG are included in the analysis; however, cancers with fewer than 10 samples were removed from the main figure and included in FIG. 6D.

FIG. 2 show mutational processes underlying clustered events. Each circle represents the activity of a signature for a given cancer type, where the radius of the circle determines the proportion of samples with greater than a given number of mutations specific to each subclass, while the color reflects the median number of mutations per cancer type. A minimum of two samples are required per cancer type for visualization.

FIGS. 3A-3G show panorama of clustered driver mutations in human cancer. Percentage of clustered mutations (top) compared to the percentage of clustered driver events (bottom) for substitutions FIG. 3A and indels FIG. 3B. FIG. 3C: The frequency of clustered driver events across known cancer genes. The radius of the circle is proportional to the number of samples with a clustered driver mutation within a gene, while the gray scale reflects the clustered mutational burden. All clustered driver events are classified into one of the five clustered classes, with the number of clustered driver substitutions and the total number of driver substitutions shown on the right. FIG. 3D: Clustered indel drivers are displayed similar to FIG. 3C. FIG. 3E: The odds ratio of clustered substitutions (top) and indels (bottom) resulting in deleterious (n=192 clustered substitutions; n=54 clustered indels) or synonymous changes (n=5 clustered substitutions; n=5 clustered indels) within a given driver gene compared to non-clustered driver mutations (n=771 deleterious and n=237 synonymous substitutions; n=111 deleterious and n=50 synonymous indels). All events were overlapped with the PCAWG consensus list of driver events and were annotated using VEP. The odds ratios are shown with their 95% confidence intervals. FIG. 3F: Kaplan-Meier survival curves comparing the outcome of samples with clustered versus non-clustered mutations in BRAF (top), TP53 (middle), and EGFR (bottom) across TCGA cohorts. Only cohorts with >5 samples harboring a clustered mutation within the given gene were included. FIG. 3G: Kaplan-Meier survival curves comparing the outcome of samples with clustered versus non-clustered mutations in the same genes across the MSK-IMPACT cohort. The log 10 (Hazards ratios) are shown with their 95% confidence intervals in FIGS. 3F and 3G. Cox regressions were corrected for age (TCGA only), mutational burden, and cancer type (see Experiment No. 1, infra.). P-values were calculated in FIGS. 3A, 3B and 3E using a two-tailed Fisher's exact test and corrected for multiple hypothesis testing.

FIGS. 4A-4F show kataegic events co-locate with most forms of structural variations. FIG. 4A: Proportion of all kataegic events per cancer type overlapping different amplifications or structural variations. FIG. 4B: The distance to the nearest breakpoint for all kataegic mutations (teal), kyklonas (gold), and non-clustered mutations (red). Kataegic distances were modeled as a Gaussian mixture with three components (blue line). FIG. 4C: Left: Volcano plot depicting samples that are statistically enriched for kyklonas (red; q-values from an FDR-corrected Z-test). Middle left: The proportion of samples with ecDNA co-occurring with kataegis. Middle right: The mutational spectrum of all kyklonas. Right: The proportion of kyklonic events attributed to SBS2 and SBS13. Cosine similarity was calculated between the kyklonic and the reconstructed spectra composed using SBS2 and SBS13 (p-value from a Z-score test). FIG. 4D: Rainfall plots illustrating the IMD distribution for a given sample with the genomic locations of ecDNA breakpoints (gray scale). FIG. 4E: YTCA versus RTCA enrichments per sample with kyklonas, where YTCA and RTCA enrichment is suggestive of higher APOBEC3A or APOBEC3B activity, respectively. Genic mutations were divided into transcribed (template strand) and coding mutations. The RTCA/YTCA fold enrichments were compared to ones of non-clustered mutations. FIG. 4F: Relative expression of APOBEC3A and APOBEC3B in (Left) samples harboring ecDNA (n=157) compared to samples without ecDNA (n=1,364) and (Right) in samples with ecDNA that have kyklonas (n=59) compared to samples without kyklonas (n=98). Expression values were normalized using FPKM and upper quartile normalization obtained from the PCAWG release. P-values in FIGS. 4E and 4F derived using a two-tailed Mann-Whitney U-test and FDR corrected using the Benjamini-Hochberg procedure. For each boxplot, the middle line reflects the median, the lower and upper bounds of the box correspond to the first and third quartiles, and the lower and upper whiskers extend from the box by 1.5× the inter-quartile range.

FIGS. 5A-5E show recurrent APOBEC3 hypermutation of ecDNA. FIG. 5A: The number of clustered events overlapping a single amplicon or structural variation (SV) event; each dot represents an amplicon or SV (n=84 circular; n=275 linear; n=111 heavily rearranged; n=62 BFB; and n=11,139 SV). A 10 kb window was used to determine the co-occurrence of kataegis with SV breakpoints (**q-values <0.01; ****q-values <0.0001). FIG. 5B: Left: As shown in gray scale, normalized distributions of the variant allele frequencies (VAFs) for all clustered mutations excluding kataegis, all non-ecDNA kataegis, and kyklonas. Right: Normalized VAF distributions for kyklonic-ecDNA harboring cancer genes and for kyklonic-ecDNA without cancer genes. FIG. 5C: The frequency of recurrence for all kataegis and kyklonas using a sliding genomic window of 10 Mb. FIG. 5D: The number of kyklonic events and kyklonic mutations per ecDNA region containing cancer genes (n=137) or not containing cancer genes (n=134; left and right; respectively). FIG. 5E: The total number of clustered and kataegic mutations found in samples with ecDNAs containing cancer genes (n=67 samples) compared to samples with ecDNAs without any cancer genes (n=44; left and right; respectively). P-values in FIGS. 5A, 5D, and 5E derived using a two-tailed Mann-Whitney U-test and FDR corrected using the Benjamini-Hochberg procedure. For data represented as boxplots in FIGS. 5A, 5D, and 5E the middle line reflects the median, the lower and upper bounds of the box correspond to the first and third quartiles, and the lower and upper whiskers extend from the box by 1.5× the inter-quartile range (IQR).

FIGS. 6A-6K show identification and clinical associations of clustered events. FIG. 6A: Schematic depiction for separating clustered mutations for a sample. FIG. 6B: Subclassification of clustered substitutions and indels. Expected IMD derived using steps 2 and 3 (panel a). FIG. 6C: Distribution of indels present in a single clustered event. FIG. 6D: Distribution of clustered substitutions (left) and indels (right) across cancers with less than 10 samples subclassified into different categories. FIG. 6E: Correlations between tumor mutational burden (TMB) of each sample, the TMB within the exome, or the TMB for each class of clustered substitutions (left) and indels (right). FIG. 6F: Distribution of variant allele frequencies for all clustered substitution classes (left; DBS: 1,215 samples; MBS: 851; omikli: 1,466; kataegis: 1,108; other: 335) with the average fold enrichment compared against non-clustered mutations (right). For each boxplot, the middle line reflects the median, the lower and upper bounds correspond to the first and third quartiles, and the lower and upper whiskers extend from the box by 1.5× the inter-quartile range (IQR). FIG. 6G: Kaplan-Meier curves between samples with high (top 80^{th} percentile) and low (bottom 20th percentile) clustered substitution (left) or indel (right) burdens in PCAWG ovarian cancer.

FIG. 6H: Cox regressions performed for PCAWG cancer types while correcting for age (n=20 upper and n=21 lower clustered substitutions; n=49 upper and n=49 lower clustered indels). FIG. 6I: Kaplan-Meier survival curves for TCGA cancer types with a differential patient outcome associated with the detection of any clustered mutations. Cox regressions performed for TCGA samples while correcting for age (FIG. 6J) and total mutational burden (FIG. 6K) (OV: n=111 upper, n=159 lower clustered substitutions; UCEC: n=322 upper, n=64 lower; ACC: n=24 upper, n=67 lower). PCAWG ovarian cancers were included in k). Center of measure for each Cox regression reflects the log₁₀(Hazards ratios) with the 95% confidence intervals in FIGS. 6H-6K).

FIGS. 7A-7E show mutational processes of clustered driver events. FIG. 7A: The percentage of clustered driver substitutions and indels within each cancer type. All samples 2,583 whole-genome sequenced samples from PCAWG with a detected driver event are included; however, cancer types with fewer than 10 samples are not presented. FIG. 7B: The proportion of clustered driver mutations per cancer gene compared between oncogenes (n=19 genes) versus tumor suppressor genes (n=30 genes) and genes with high numbers of isoforms (n=17) versus genes with low numbers of isoforms (n=23; upper and lower quartiles of isoforms across all cancer drivers). FIG. 7C: The proportion of clustered driver mutations for a given subclass per cancer gene compared between oncogenes (n=17 genes with clustered substitutions and n=13 with for clustered indels) versus tumor suppressor genes (n=28 genes with clustered substitutions and n=70 genes with clustered indels). FIG. 7D: The relative expression of driver genes harboring clustered versus non-clustered events. All expression values were normalized using FPKM normalization and upper quartile normalization obtained from the official PCAWG release and were subsequently normalized using the average expression of the wild-type gene. A value of 1 (dashed lined) reflects no difference in expression compared to the wild-type gene. FIG. 7E: The proportional activity of mutational signatures contributing to clustered driver events within each subclass. Multi-base substitutions (MBSs) did not contribute to any reported driver events. For analyses in b-d) p-values were generated using a two-tailed Mann-Whitney U test (*P<0.05; p=0.03 for STAT6; p=0.04 for CTNNB1; p=0.02 for BTG1). For each boxplot, the middle line reflects the median, the lower and upper bounds of the box correspond to the first and third quartiles, and the lower and upper whiskers extend from the box by 1.5× the inter-quartile range (IQR)

FIGS. 8A-8E show recurrent mutagenesis and functional effects of kyklonas. FIG. 8A: The total number of recurrently mutated ecDNA displayed as a proportion of the total number of ecDNA with kyklonas for a given cancer type. The total number of ecDNA with kyklonas are displayed above each bar plot for each cancer type. All ecDNA with recurrent hypermutation were considered enriched for kyklonic events after correcting for multiple hypothesis testing (Z-score test; q-values <0.05). FIG. 8B: Proportion of samples harboring ecDNA divided exclusively into those with co-occurring kataegis, no kataegis overlap, and no detected kataegis across the entire genome. The number of samples included in each cancer type are listed. For certain cancer types, as few as a single sample may represent the entire proportional breakdown (e.g., Bone-Osteosarc or Bone-Epith). FIG. 8C: A single sarcoma genome and (FIG. 8D) a single head squamous cell carcinoma genome depicting the overlap of kataegis with ecDNA regions displayed as a rainfall (top left) with a single zoomed in ecDNA represented using a circos plot (top right). Bottom: Two regions of the ecDNA with overlapping kyklonic events. Variant allele frequencies are shown per event (orange). FIG. 8E: Kyklonic substitutions resulting in recurrent coding mutations within known cancer genes.

FIGS. 9A-9B show determining the number of mutations differentiating between omikli and kataegis. FIG. 9A: Modeling the number of mutations per event using a mixture of two Poisson distributions. The first component, representative of omikli, has an average IMD of 2.1, while the second component, representative of kataegis, has an average IMD of 4.4. The estimated contribution of mutations of each component are depicted as bars for each corresponding event size FIG. 9B: The distribution of IMDs per event across different sized events (n=199,912 events with 2 mutations; n=35,576 events with 3 mutations; n=15,320 events with 4 mutations; n=9,613 events with 5 mutations). The chosen cutoff between omikli and kataegis was four mutations. For each boxplot, the middle line reflects the median, the lower and upper bounds of the box correspond to the first and third quartiles, and the lower and upper whiskers extend from the box by 1.5× the inter-quartile range (IQR).

FIG. 10 shows the distribution of low confidence clustered indels. The number of clustered indels falling within regions of the genome with low mapping scores consists of approximately 1% of all clustered indels. Within these 1% of mutations with low mapping scores, only 30% of events have an inter-mutational distance less than 10 (0.3% of all clustered indels), while indels of lbp falling within low mapping regions comprise only 0.5% of all clustered indels.

FIGS. 11A-11B are Kaplan-Meier survival curves comparing the outcome of samples with clustered versus non-clustered mutations in the same genes across the MSK-MET cohort comprised of targeted sequencing from both primary (FIG. 11A) and metastatic (FIG. 11B) cancers. The log 10-transformed hazards ratios (log 10 (HR)) are shown with their 95% confidence intervals. Cox regressions were corrected for age, tumor mutational burden, and gender. Each comparison was derived using a single cancer type.

FIGS. 12A-12B show de novo signatures of doublet-base (DBS) and multi-base (MBS) signatures. FIG. 12A: The activity of DBS de novo signatures (top) and the corresponding signatures extracted from prostate, skin, stomach, and uterine cancers that could not be accurately reconstructed using known COSMIC mutational signatures (bottom). FIG. 12B: The activity of MBS de novo signatures (top) and the corresponding signatures extracted from colon, esophagus, and head and neck cancers that could not be accurately reconstructed using known COSMIC mutational signatures (bottom).

FIGS. 13A-13D show experimental validation and epidemiological associations of clustered mutational processes. FIG. 13A: Experimental validation of three omikli processes. Specifically, APOBEC3-associated omikli were validated using a clonally expanded BT-474 breast cancer cell line (top), omikli events resulting from exposure to benzo[a]pyrene were validated using iPSC cells (middle), and omikli events resulting from exposure to ultraviolet light were validated using iPSC cells (bottom). FIG. 13B: Mutational processes of strand-coordinated kataegic events. FIG. 13C: Epidemiological associations comparing the ratio of clustered tumor mutational burden (TMB) to the total TMB for a given sample between: drinkers (n=25) and non-drinkers (n=61); smokers (n=68) and non-smokers (n=11); homologous-recombination deficient (HR-deficient; n=25) and homologous-recombination proficient samples (HR-proficient; n=64). For each boxplot, the middle line reflects the median, the lower and upper bounds of the box correspond to the first and third quartiles, and the lower and upper whiskers extend from the box by 1.5× the inter-quartile range (IQR). P-values were calculated using a two-tailed Mann-Whitney U test. FIG. 13D: Mutational processes of clustered events with inconsistent variant allele frequency (VAFs) classified as other clustered substitutions. A minimum of two samples are required per cancer type for visualization.

FIGS. 14A-14B are examples of clustered mutational signatures. (FIG. 14A) Two samples depicting the intra-mutational distance (IMD) distributions of substitutions across genomic coordinates, where each dot represents the minimum distance to adjacent mutations for a selected mutation colored based upon the corresponding subclassification of event (rainfall plot; left). The red lines depict the sample-dependent IMD threshold for each sample. Specific clustered mutations may be above this threshold based upon corrections for regional mutation density. The mutational spectra for the different catalogs of clustered and non-clustered substitutions for each sample (right; MBS are not shown). (FIG. 14B) Two samples illustrating the IMD distributions of indels across the given genomes, with the IMD indel thresholds shown in red (left). The non-clustered and clustered indel catalogs for each sample (right).

FIGS. 15A-15D_show clustered events and structural variations. FIG. 15A: The proportion of all clustered events co-locating with structural variations across all cancer types (left) and across each cancer type (right). FIG. 15B: The distance to the nearest structural variation for each class of clustered mutations (gray scale), and non-clustered mutations (red). The distribution for each class of clustered events were modeled using a Gaussian mixture (gray scale). DBSs and MBSs were modeled using a single distribution, while omikli, other, and indels were modeled using two components reflecting the minimal distribution of overlap with structural variations. FIG. 15C: The mutational signatures active in ecDNA clustered events. FIG. 15D: YTCA versus RTCA enrichments per sample within non-ecDNA kataegis (top) and non-SV associated kataegis (bottom), where YTCA and RTCA enrichment is suggestive of APOBEC3A or APOBEC3B activity, respectively. Genic mutations were divided into transcribed (template strand) and coding mutations. The RTCA/YTCA fold enrichments were compared to the fold enrichments of non-clustered mutations (p-values calculated using two-tailed Mann-Whitney U-tests and corrected for multiple hypothesis testing using the Benjamini-Hochberg false discovery rate procedure).

FIGS. 16A-16E show validation of APOBEC3 hypermutation of ecDNA in three independent cohorts. FIG. 16A: Distribution of clustered substitutions (left) and clustered indels (right) across three validation cohorts. Clustered substitutions were subclassified into doublet-base substitutions, multi-base substitutions, omikli, kataegis, and other clustered mutations. Top: Each black dot represents a single cancer genome. Gray scaled bars reflect the median clustered tumor mutational burden (TMB) and the percentage of clustered mutations contributing to the overall TMB of a given sample for each cancer type. Middle: The proportion of each subclass of clustered events for a given cancer type with the total number of samples having at least a single clustered event over the total number of samples within a given cancer cohort. Bottom: Percentage of clustered mutations compared to the percentage of clustered driver events for substitutions (left) and indels (right). P-values were calculated using a Fisher's exact test and corrected for multiple hypothesis testing using Benjamini-Hochberg false discovery rate procedure. FIG. 16B: Left: The mutational spectrum of all kyklonas across the validation cohorts. Right: The proportion of kyklonic events attributed to SBS2 and SBS13 (p-value determined using a Z-score test). FIG. 16C: The proportion of samples with ecDNA that co-occur with kataegis, do not co-occur with kataegis, or do not have any detected kataegic activity across each cohort. FIG. 16D: YTCA versus RTCA enrichments per sample with kyklonas, where YTCA and RTCA enrichment is suggestive of higher APOBEC3A or APOBEC3B activity, respectively. The RTCA/YTCA fold enrichments were compared to the fold enrichments of non-clustered mutations (p-values calculated using a two-tailed Mann-Whitney U-test). FIG. 16E: The proportion of ecDNA with kyklonas that harbor multiple kyklonic events. The total number of ecDNA with kyklonas are displayed above each bar plot for each cancer type.

FIGS. 17A-17B show Kyklonas occur distally from structural breakpoints across three independent cohorts. FIG. 17A: The distance to the nearest breakpoint for all kataegic mutations (gray scale), kyklonas (gray scale), and non-clustered mutations (gray scale) across the three validation cohorts. FIG. 17B: Distances to the nearest SV breakpoints were normalized by calculating the expected distance a mutation would fall from a breakpoint given the number of breakpoints detected per chromosome and the overall length of the chromosome across the validation cohorts (gray scale) and PCAWG (gray scale). A value of 1 (dashed line) reflects a distance that one would expect based on the random placement of a mutation across the chromosome, while a value less than 1 reflects a mutation occurring closer than what is expected by random chance. The distributions of kataegic mutations were modelled using Gaussian mixture models (gray scale lines) with an automatic selection criterion for the number of components using the minimum Bayesian information criteria (BIC).

FIGS. 18A-18C are examples of kyklonas in three independent cohorts. FIG. 18A: A single undifferentiated sarcoma genome depicting the overlap of kataegis with ecDNA regions displayed as a rainfall (left) with a single zoomed in ecDNA represented using a circos plot (middle). The outer track of the circos plot represents the reference genome of the ecDNA with proximal known cancer driver genes. The middle track reflects a circular rainfall plot where each dot represents the IMD around a single mutation colored based on the substitution change. The innermost track shows the average variant allele frequency (VAF) for each kyklonic event. Right: Two smaller regions of the selected ecDNA including a single kyklonic event within ZNF536 region resulting in a plethora of missense and stop-gained mutations, and a single kyklonic event within a promoter flanking with the average VAFs per event. FIG. 18B: A single lung adenocarcinoma genome depicting the overlap of kataegis with ecDNA regions (left) with a single zoomed in ecDNA harboring TBC1D15 and two distinct kyklonic events represented using a circos plot (middle). Right: Two kyklonic events overlapping an upstream region and TBC1D15. FIG. 18C: A single esophageal squamous cell carcinoma genome depicting the overlap of kataegis with ecDNA regions (left) with a single zoomed in ecDNA harboring PRKAA2 and DAB1 and three distinct kyklonic events (middle). Right: Two kyklonic events overlapping DAB1.

### DETAILED DESCRIPTION OF THE DISCLOSURE

It is to be understood that the present disclosure is not limited to particular aspects described, as such may, of course, vary. It is also to be understood that the terminology used herein is for the purpose of describing particular aspects only, and is not intended to be limiting, since the scope of the present disclosure will be limited only by the appended claims.

Unless defined otherwise, all technical and scientific terms used herein have the same meanings as commonly understood by one of ordinary skill in the art to which this technology belongs. Although any methods and materials similar or equivalent to those described herein can be used in the practice or testing of the present technology, the preferred methods, devices and materials are now described. All technical and patent publications cited herein are incorporated herein by reference in their entirety. Nothing herein is to be construed as an admission that the present technology is not entitled to antedate such disclosure by virtue of prior invention.

The practice of the present technology will employ, unless otherwise indicated, conventional techniques of tissue culture, immunology, molecular biology, microbiology, cell biology, and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

It is to be inferred without explicit recitation and unless otherwise intended, that when the present technology relates to a polypeptide, protein, polynucleotide or antibody, an equivalent or a biologically equivalent of such is intended within the scope of the present technology.

### Definitions

As used in the specification and claims, the singular form “a”, “an”, and “the” include plural references unless the context clearly dictates otherwise. For example, the term “a cell” includes a plurality of cells, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. For example, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions disclosed herein. Aspects defined by each of these transition terms are within the scope of the present disclosure.

As used herein, the term “animal” refers to living multi-cellular vertebrate organisms, a category that includes, for example, mammals and birds. The term “mammal” includes both human and non-human mammals.

In one aspect, the term “equivalent” or “biological equivalent” of an antibody means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody.

In one aspect, the term “equivalent” of “chemical equivalent” of a chemical means the ability of the chemical to selectively interact with its target protein, DNA, RNA or fragment thereof as measured by the inactivation of the target protein, incorporation of the chemical into the DNA or RNA or other suitable methods. Chemical equivalents include, but are not limited to, those agents with the same or similar biological activity and include, without limitation a pharmaceutically acceptable salt or mixtures thereof that interact with and/or inactivate the same target protein, DNA, or RNA as the reference chemical.

The term “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions and insertions of nucleotides. An allele of a gene can also be a form of a gene containing a mutation.

The term “genetic marker” refers to an allelic variant of a polymorphic region of a gene of interest and/or the expression level of a gene of interest.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic region can be a single nucleotide, the identity of which differs in different alleles.

The term “genotype” refers to the specific allelic composition of an entire cell or a certain gene and in some aspects a specific polymorphism associated with that gene, whereas the term “phenotype” refers to the detectable outward manifestations of a specific genotype.

The term “isolated” as used herein refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis.

As used herein, “aggressive therapy” or “aggressive chemotherapy” may refer to any one or a combination of therapeutic cancer therapies, including but not limited to any form of chemical drug therapy meant to destroy rapidly growing/proliferating cancer cells within the body. “Aggressive chemotherapy” refers to any therapy that may extend beyond the first line of treatment or the standard therapeutic regimen for any particular cancer or tumor. “Aggressive chemotherapy” may include, but is not limited to, adoptive cell therapy, immune checkpoint blockades including PD1, PD-L1, and CTLA4, pretargeted radioimmunotherapy, oncolytic viral therapy, or cancer vaccines.

When the disease is cancer, the following clinical endpoints are non-limiting examples of treatment: (1) elimination of a cancer in a subject or in a tissue/organ of the subject or in a cancer loci; (2) reduction in tumor burden (such as number of cancer cells, number of cancer foci, number of cancer cells in a foci, size of a solid cancer, concentrate of a liquid cancer in the body fluid, and/or amount of cancer in the body); (3) stabilizing or delay or slowing or inhibition of cancer growth and/or development, including but not limited to, cancer cell growth and/or division, size growth of a solid tumor or a cancer loci, cancer progression, and/or metastasis (such as time to form a new metastasis, number of total metastases, size of a metastasis, as well as variety of the tissues/organs to house metastatic cells); (4) less risk of having a cancer growth and/or development; (5) inducing an immune response of the patient to the cancer, such as higher number of tumor-infiltrating immune cell, higher number of activated immune cells, or higher number cancer cell expressing an immunotherapy target, or higher level of expression of an immunotherapy target in a cancer cell; (6) higher probability of survival and/or increased duration of survival, such as increased overall survival (OS, which may be shown as 1-year, 2-year, 5-year, 10-year, or 20-year survival rate), increased progression free survival (PFS), increased disease free survival (DFS), increased time to tumor recurrence (TTR) and increased time to tumor progression (TTP). In some embodiments, the subject after treatment experiences one or more endpoints selected from tumor response, reduction in tumor size, reduction in tumor burden, increase in overall survival, increase in progression free survival, inhibiting metastasis, improvement of quality of life, minimization of drug-related toxicity, and avoidance of side-effects (e.g., decreased treatment emergent adverse events). In some embodiments, improvement of quality of life includes resolution or improvement of cancer-specific symptoms, such as but not limited to fatigue, pain, nausea/vomiting, lack of appetite, and constipation; improvement or maintenance of psychological well-being (e.g., degree of irritability, depression, memory loss, tension, and anxiety); improvement or maintenance of social well-being (e.g., decreased requirement for assistance with eating, dressing, or using the restroom; improvement or maintenance of ability to perform normal leisure activities, hobbies, or social activities; improvement or maintenance of relationships with family). In some embodiments, improved patient quality of life that is measured qualitatively through patient narratives or quantitatively using validated quality of life tools known to those skilled in the art, or a combination thereof. Additional non-limiting examples of endpoints include reduced hospital admissions, reduced drug use to treat side effects, longer periods off-treatment, and earlier return to work or caring responsibilities. In one aspect, prevention or prophylaxis is excluded from treatment.

As used herein, immune cells are cells of the immune system, including but not limited to lymphocytes (such as, T-cells, B-cells, natural killer (NK) cells, and natural killer T (NKT) cells), myeloid-derived cells (such as granulocytes (basophils, eosinophils, neutrophils, mast cells), monocytes, macrophages, and dendritic cells (DC)). T cells are divided into two broad categories: CD8+ T cells or CD4+ T cells, based on which protein is present on the cell's surface. CD8+ T cells also are called cytotoxic T cells or cytotoxic lymphocytes (CTLs). The four major CD4+ T-cell subsets are TH1, TH2, TH17, and Treg, with “TH” referring to “T helper cell.” T cells may also refer to gamma delta T cell. Dendritic cells (DC) are an important antigen-presenting cell (APC), and they also can develop from monocytes. In some embodiments, the immune cells refer to a killer cell, including but not limited to: a cytotoxic T cell, a gamma delta T cell, a NK cell and a NK-T cell. In one embodiment, the immune cell is a CD45+ cell.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has or is diagnosed of having or is suspected of having a cancer. The subject can be a male or female.

In certain embodiments, the terms “disease” “disorder” and “condition” are used interchangeably herein, referring to a cancer, a status of being diagnosed with a cancer, or a status of being suspect of having a cancer. “Cancer”, which is also referred to herein as “tumor”, is a known medically as an uncontrolled division of abnormal cells in a part of the body, benign or malignant. In one embodiment, cancer refers to a malignant neoplasm, a broad group of diseases involving unregulated cell division and growth, and invasion to nearby parts of the body. Non-limiting examples of cancers include carcinomas, sarcomas, leukemia and lymphoma, e.g., colon cancer, colorectal cancer, rectal cancer, gastric cancer, esophageal cancer, head and neck cancer, breast cancer, brain cancer, lung cancer, stomach cancer, liver cancer, gall bladder cancer, or pancreatic cancer. In one embodiment, the term “cancer” refers to a solid tumor, which is an abnormal mass of tissue that usually does not contain cysts or liquid areas, including but not limited to, sarcomas, carcinomas, and certain lymphomas (such as Non-Hodgkin's lymphoma). In another embodiment, the term “cancer” refers to a liquid cancer, which is a cancer presenting in body fluids (such as, the blood and bone marrow), for example, leukemias (cancers of the blood) and certain lymphomas.

Additionally or alternatively, a cancer may refer to a local cancer (which is an invasive malignant cancer confined entirely to the organ or tissue where the cancer began), a metastatic cancer (referring to a cancer that spreads from its site of origin to another part of the body), a non-metastatic cancer, a primary cancer (a term used describing an initial cancer a subject experiences), a secondary cancer (referring to a metastasis from primary cancer or second cancer unrelated to the original cancer), an advanced cancer, an unresectable cancer, or a recurrent cancer. As used herein, an advanced cancer refers to a cancer that had progressed after receiving one or more of: the first line therapy, the second line therapy, or the third line therapy.

The term “chemotherapy” encompasses cancer therapies that employ chemical or biological agents or other therapies, such as radiation therapies, e.g., a small molecule drug or a large molecule, such as antibodies, immunotherapies, RNAi and gene therapies. Non-limiting examples of chemotherapies are provided below. It should be understood, although not always explicitly stated, that when a particular therapy is noted, the scope of the disclosure includes equivalents unless excluded.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

As used herein, the term “administration” and “administering” are used to mean introducing an agent into a subject. Routes of administration include, but are not limited to, oral (such as a tablet, capsule or suspension), topical, transdermal, intranasal, vaginal, rectal, subcutaneous intravenous, intravenous, intraarterial, intramuscular, intraosseous, intraperitoneal, intraocular, subconjunctival, sub-Tenon's, intravitreal, retrobulbar, intracameral, intratumoral, epidural and intrathecal.

An “immunotherapy agent” means a type of cancer treatment which uses a patient's own immune system to fight cancer, including but not limited to a physical intervene, a chemical substance, a biological molecule or particle, a cell, a tissue or organ, or any combinations thereof, enhancing or activating or initiating a patient's immune response against cancer. Non-limiting examples of immunotherapy agents include antibodies, immune regulators, checkpoint inhibitors, an antisense oligonucleotide (ASO), a RNA interference (RNAi), a Clustered Regularly Interspaced Short Palindromic Repeat (CRISPR) system, a viral vector, an anti-cancer cell therapy (e.g., transplanting an anti-cancer immune cell optionally amplified and/or activated in vivo, or administering an immune cell expressing a chimeric antigen receptor (CAR)), a CAR therapy, and cancer vaccines. As used herein, unless otherwise specified, an immunotherapy agent is not an inhibitor of thymidylate biosynthesis, or an anthracycline or other topoisomerase II inhibitor. As used herein, immune checkpoint refers to a regulator and/or modulator of the immune system (such as an immune response, an anti-tumor immune response, a nascent anti-tumor immune response, an anti-tumor immune cell response, an anti-tumor T cell response, and/or an antigen recognition of T cell receptor in the process of immune response). Their interaction activates either inhibitory or activating immune signaling pathways. Thus a checkpoint may contain one of the two signals: a stimulatory immune checkpoint that stimulates an immune response, and an inhibitory immune checkpoint inhibiting an immune response. In some embodiments, the immune checkpoint is crucial for self-tolerance, which prevents the immune system from attacking cells indiscriminately. However, some cancers can protect themselves from attack by stimulating immune checkpoint targets. In some embodiments, the immune checkpoints are present on T cells, antigen-presenting cells (APCs) and/or tumor cells.

One target of an immunotherapy agent is a tumor-specific antigen while the immunotherapy directs or enhances the immune system to recognize and attack tumor cells. Non-limiting examples of such agent includes a cancer vaccine presenting a tumor-specific antigen to the patient's immune system, a monoclonal antibody or an antibody-drug conjugate specifically binding to a tumor-specific antigen, a bispecific antibody specifically binding to a tumor-specific antigen and an immune cell (such as a T-cell engager or a NK-cell engager), an immune cell (such as a killer cell) specifically binding to a tumor-specific antigen (such as a CAR-T cell, a CAR-NK cell, and a CAR-NKT cell), a polynucleotide (or a vector comprising the same) transfecting/transducing an immune cell to express an tumor-specific antibody of an antigen binding fragment thereof (such as a CAR), or a polynucleotide (or a vector comprising the same) transfecting/transducing a cancer cell to express an antigen or a marker which can be recognized by an immune cell.

Another exemplified target is an inhibitory immune checkpoint which suppresses the nascent anti-tumor immune response, such as A2AR, B7-H3, B7-H4, BTLA, CTLA-4, CTLA-4/B7-1/B7-2, IDO, KIR, LAG3, NOX2, PD-1, PD-L1 and TIM-3, VISTA, SIGLEC7 (Sialic acid-binding immunoglobulin-type lectin 7, also designated as CD328) and SIGLEC9 (Sialic acid-binding immunoglobulin-type lectin 9, also designated as CD329). Non-limiting examples of such agent includes an antagonist or inhibitor of an inhibitory immune checkpoint, an agent reducing the expression and/or activity of an inhibitory immune checkpoint (such as via an antisense oligonucleotide (ASO), a RNA interference (RNAi), or a Clustered Regularly Interspaced Short Palindromic Repeat (CRISPR) system), an antibody or an antibody-drug conjugate or a ligand specifically binding to and reducing (or inhibiting) the activity of an inhibitory immune checkpoint, an immune cell with reduced (or inhibited) an inhibitory immune checkpoint (and optionally specifically binding to a tumor-specific antigen, such as a CAR-T cell, a CAR-NK cell, and a CAR-NKT cell), and a polynucleotide (or a vector comprising the same) transfecting/transducing an immune cell or a cancer cell to reduce or inhibit an inhibitory immune checkpoint thereof. Reducing expression or activity of such inhibitory immune checkpoint enhances immune response of a patient to a cancer.

A further possible immunotherapy target is a stimulatory checkpoint molecule (including but not limited to 4-1BB, CD27, CD28, CD40, CD122, CD137, OX40, GITR and ICOS), wherein the immunotherapy agent actives or enhances the anti-tumor immune response. Non-limiting examples of such agent includes an agonist of a stimulatory checkpoint, an agent increasing the expression and/or activity of a stimulating immune checkpoint, an antibody or an antibody-drug conjugate or a ligand specifically binding to and activating or enhancing the activity of a stimulating immune checkpoint, an immune cell with increased expression and/or activity of a stimulating immune checkpoint (and optionally specifically binding to a tumor-specific antigen, such as a CAR-T cell, a CAR-NK cell, and a CAR-NKT cell), and a polynucleotide (or a vector comprising the same) transfecting/transducing an immune cell or a cancer cell to express a stimulating immune checkpoint thereof.

Additional or alternative targets may be utilized by an immunotherapy agent, such as an immune regulating agent, including but not limited to, an agent activating an immune cell, an agent recruiting an immune cell to a cancer or a cancer cell, or an agent increasing immune cell infiltrated into a solid tumor and/or a cancer loci. Non-limiting examples of such agent is an immune regulator or a variant, a mutant, a fragment, an equivalent thereof.

In some embodiments, an immunotherapy agent utilizes one or more targets, such as a bispecific T cell engager, a bispecific NK cell engager, or a CAR cell therapy. In some embodiments, the immunotherapy agent targets one or more immune regulatory or effector cells.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits, rat, canine, donkey, mice, camelids (such as dromedaries, llamas, and alpacas), as well as non-mammalian species, such as shark immunoglobulins. Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴M⁻¹ greater or at least 10⁵ M⁻¹ greater than a binding constant for other molecules in a biological sample). The term “antibody” also includes genetically engineered forms such as chimeric antibodies (for example, murine or humanized non-primate antibodies), heteroconjugate antibodies (such as, bispecific antibodies). See also, Pierce Catalog and Handbook, 1994-1995 (Pierce Chemical Co., Rockford, Ill.); Owen et al., Kuby Immunology, 7^{th} Ed., W.H. Freeman & Co., 2013; Murphy, Janeway's Immunobiology, 8^{th} Ed., Garland Science, 2014; Male et al., Immunology (Roitt), 8^{th} Ed., Saunders, 2012; Parham, The Immune System, 4^{th} Ed., Garland Science, 2014. The term “antibody” includes any protein or peptide containing molecule that comprises at least a portion of an immunoglobulin molecule, such as the whole antibody and any antigen binding fragment or a single chain thereof. The terms “antibody,” “antibodies” and “immunoglobulin” also include immunoglobulins of any isotype, fragments of antibodies which retain specific binding to antigen, including, but not limited to, Fab, Fab′, F(ab)₂, Fv, scFv, dsFv, Fd fragments, dAb, VH, VL, VhH, and V-NAR domains; minibodies, diabodies, triabodies, tetrabodies and kappa bodies; multispecific antibody fragments formed from antibody fragments and one or more isolated. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, at least one portion of a binding protein, chimeric antibodies, humanized antibodies, single-chain antibodies, and fusion proteins comprising an antigen-binding portion of an antibody and a non-antibody protein. The variable regions of the heavy and light chains of the immunoglobulin molecule contain a binding domain that interacts with an antigen. The constant regions of the antibodies (Abs) may mediate the binding of the immunoglobulin to host tissues. The antibodies can be polyclonal, monoclonal, multispecific (e.g., bispecific antibodies), and antibody fragments, so long as they exhibit the desired biological activity.

As used herein, the term “monoclonal antibody” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

In some embodiments, the antibody is a bispecific immune cell engager, referring to a bispecific monoclonal antibody that is capable of recognizing and specifically binding to a tumor antigen (such as CD19, EpCAM, MCSP, HER2, EGFR or CS-1) and an immune cell, and directing an immune cell to cancer cells, thereby treating a cancer. Non-limiting examples of such antibody include bispecific T cell engager, bispecific cytotoxic T lymphocytes (CTL) engager, and bispecific NK cell engager. In one embodiment, the engager is a fusion protein consisting of two single-chain variable fragments (scFvs) of different antibodies. Additionally or alternatively, the immune cell is a killer cell, including but not limited to: a cytotoxic T cell, a gamma delta T cell, a NK cell and a NK-T cell.

As used herein, the term “antigen binding domain” refers to any protein or polypeptide domain that can specifically bind to an antigen target.

The term “chimeric antigen receptor” (CAR), as used herein, refers to a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” or “intracellular signaling domain” means any oligopeptide or polypeptide known to function as a domain that transmits a signal to cause activation or inhibition of a biological process in a cell. In certain embodiments, the intracellular domain may comprise, alternatively consist essentially of, or yet further comprise one or more costimulatory signaling domains in addition to the primary signaling domain. The “transmembrane domain” means any oligopeptide or polypeptide known to span the cell membrane and that can function to link the extracellular and signaling domains. A chimeric antigen receptor may optionally comprise a “hinge domain” which serves as a linker between the extracellular and transmembrane domains.

As used herein, a CAR therapy may refer to administrating an immune cell expressing a CAR into a subject as well as contacting a vector expressing a CAR in an immune cell (such as in vivo).

As used herein, the term “NK cell,” also known as natural killer cell, refers to a type of lymphocyte that originates in the bone marrow and play a critical role in the innate immune system. NK cells provide rapid immune responses against viral-infected cells, tumor cells or other stressed cell, even in the absence of antibodies and major histocompatibility complex on the cell surfaces. NK cells for using in a cell therapy and/or a CAR therapy may either be isolated or obtained from a commercially available source. Non-limiting examples of commercial NK cell lines include lines NK-92 (ATCC® CRL-2407™), NK-92 MI (ATCC® CRL-2408™). Further examples include but are not limited to NK lines HANK1, KHYG-1, NKL, NK—YS, NOI-90, and YT. Non-limiting exemplary sources for such commercially available cell lines include the American Type Culture Collection, or ATCC, (http://www.atcc.org/) and the German Collection of Microorganisms and Cell Cultures (https://www.dsmz.de/).

As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells for using in a cell therapy and/or a CAR therapy may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™), TALL-104 cytotoxic human T cell line (ATCC #CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4; 11 (ATCC CRL-1873), CCRF-CEM (ATCC CRM-CCL-119); and cutaneous T-cell lymphoma lines, e.g., HuT78 (ATCC CRM-TIB-161), MJ[G11] (ATCC CRL-8294), HuT102 (ATCC TIB-162). Null leukemia cell lines, including but not limited to REH, NALL-1, KM-3, L92-221, are another commercially available source of immune cells for using in a CAR therapy, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (http://www.atcc.org/) and the German Collection of Microorganisms and Cell Cultures (https://www.dsmz.de/).

As used herein, a “tumor-specific antigen” refers to an antigenic substance produced in tumor cells, capable of triggering an immune response in a subject. In some embodiments, such tumor-specific antigen is not expressed on or in a cell in the subject, which is not a cancer cell. In some embodiment, such tumor-specific antigen may still be expressed in or on some non-cancer cells. For example, a tumor-specific antigen may not be expressed on the cell surface of a non-cancer cell in the subject. In one embodiment, the tumor-specific antigen may be expressed in or on a non-cancer cell of the subject, but in a much lower level compared to a cancer cell. In another embodiment, the tumor-specific antigen may be expressed in or on a non-cancer cell of the subject which is not adjacent to a cancer or a cancer cell. Non-limiting examples of a tumor-specific antigen includes: Alphafetoprotein (AFP), Beta-2-microglobulin (B2M), Beta-human chorionic gonadotropin (Beta-hCG), Bladder Tumor Antigen (BTA), C-kit/CD117, CA15-3/CA27.29, CA19-9, CA-125, CA 27.29, Calcitonin, Carcinoembryonic antigen (CEA), Chromogranin A (CgA), Cytokeratin fragment 21-1, Des-gamma-carboxy prothrombin (DCP), Estrogen receptor (ER)/progesterone receptor (PR), Epithelial tumor antigen (ETA), Fibrin/fibrinogen, Gastrin, HE4, overexpressed HER2/neu, 5-HIAA, Lactate dehydrogenase, Melanoma-associated antigen (MAGE), MUC-1, Neuron-specific enolase (NSE), Nuclear matrix protein 22, Programmed death ligand 1 (PD-L1), Prostate-specific antigen (PSA), Prostatic Acid Phosphatase (PAP), Soluble mesothelin-related peptides (SMRP), Somatostatin receptor, Tyrosinase, Thyroglobulin, abnormal products of ras, p53, alpha folate receptor, 5T4, αvβ6 integrin, BCMA, B7-H3, B7-H6, CAIX, CD16, CD19, CD20, CD22, CD25, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvni, EGP2, EGP40, EPCAM, EphA2, EpCAM, FAP, fetal AchR, FRoc, GD2, GD3, Glypican-3 (GPC3), HL A-A 1+M AGE 1, HLA-A2+MAGE1, HL A-A3+M AGE 1, HLA-A1+NY-ESO-1, HL A-A2+NY-ESO-1, HLA-A3+NY-ESO-1, IL-11Roc, IL-13Ra2, Lambda, Lewis-Y, Kappa, Mesothelin, Mucl, Muc16, NCAM, NKG2D Ligands, NY-ESO-1, PRAME, PSCA, PSMA, RORI, SSX, Survivin, TAG72, TEMs, VEGFR2, and WT-1.

“An effective amount” or “therapeutically effect amount” intends to indicate the amount of a compound or agent administered or delivered to the patient which is most likely to result in the desired response to treatment. The amount is empirically determined by the patient's clinical parameters including, but not limited to the Stage of disease, age, gender, histology, and likelihood for tumor recurrence.

A “patient” as used herein intends an animal patient, a mammal patient or yet further a human patient. For the purpose of illustration only, a mammal includes but is not limited to a simian, a murine, a bovine, an equine, a porcine or an ovine subject. The patient can be a female or male.

The term “clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), objective response rate (RR or ORR), toxicity or side effect.

The term “suitable for a therapy” or “suitably treated with a therapy” shall mean that the patient is likely to exhibit one or more desirable clinical outcomes as compared to patients having the same disease and receiving the same therapy but possessing a different characteristic that is under consideration for the purpose of the comparison. In one aspect, the characteristic under consideration is a genetic polymorphism or a somatic mutation. In another aspect, the characteristic under consideration is expression level of a gene or a polypeptide. In one aspect, a more desirable clinical outcome is relatively higher likelihood of or relatively better tumor response such as tumor load reduction. In another aspect, a more desirable clinical outcome is relatively longer overall survival. In yet another aspect, a more desirable clinical outcome is relatively longer progression free survival or time to tumor progression. In yet another aspect, a more desirable clinical outcome is relatively longer disease free survival. In further another aspect, a more desirable clinical outcome is relative reduction or delay in tumor recurrence. In another aspect, a more desirable clinical outcome is relatively decreased metastasis. In another aspect, a more desirable clinical outcome is relatively lower relative risk. In yet another aspect, a more desirable clinical outcome is relatively reduced toxicity or side effects. In some embodiments, more than one clinical outcomes are considered simultaneously. In one such aspect, a patient possessing a characteristic, such as a genotype of a genetic polymorphism, can exhibit more than one more desirable clinical outcomes as compared to patients having the same disease and receiving the same therapy but not possessing the characteristic. As defined herein, the patient is considered suitable for the therapy. In another such aspect, a patient possessing a characteristic can exhibit one or more desirable clinical outcome but simultaneously exhibit one or more less desirable clinical outcome. The clinical outcomes will then be considered collectively, and a decision as to whether the patient is suitable for the therapy will be made accordingly, taking into account the patient's specific situation and the relevance of the clinical outcomes. In some embodiments, progression free survival or overall survival is weighted more heavily than tumor response in a collective decision making.

Response criteria can be based on the RECIST criteria (Therasse and Arbuck et al., 2000, New Guidelines to Evaluate Response to Treatment in Solid Tumors, J Natl Cancer Inst, 92:205-16). A “complete response” (CR) to a therapy refers to the clinical status of a patient with evaluable but non-measurable disease, whose tumor and all evidence of disease have disappeared following administration of the therapy. In this context, a “partial response” (PR) refers to a response that is anything less than a complete response. “Stable disease” (SD) indicates that the patient is stable following the therapy. “Progressive disease” (PD) indicates that the tumor has grown (i.e. become larger) or spread (i.e. metastasized to another tissue or organ) or the overall cancer has gotten worse following the therapy. For example, tumor growth of more than 20 percent since the start of therapy typically indicates progressive disease. “Non-response” (NR) to a therapy refers to status of a patient whose tumor or evidence of disease has remained constant or has progressed.

“Overall Survival” (OS) refers to the length of time of a cancer patient remaining alive following a cancer therapy.

“Progression free survival” (PFS) or “Time to Tumor Progression” (TTP) refers to the length of time following a therapy, during which the tumor in a cancer patient does not grow. Progression-free survival includes the amount of time a patient has experienced a complete response, partial response or stable disease.

“Disease free survival” refers to the length of time following a therapy, during which a cancer patient survives with no signs of the cancer or tumor.

“Time to Tumor Recurrence (TTR)” refers to the length of time, following a cancer therapy such as surgical resection or chemotherapy, until the tumor has reappeared (come back). The tumor may come back to the same place as the original (primary) tumor or to another place in the body.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

“Objective response rate” refers to the proportion of responders (patients with either a partial (PR) or complete response (CR) compared to nonresponders (patients with either SD or PD). Response duration can be measured from the time of initial response until documented tumor progression.

The term “identify” or “identifying” is to associate or affiliate a patient closely to a group or population of patients who likely experience the same or a similar clinical response to a therapy.

The term “selecting” a patient for a therapy refers to making an indication that the selected patient is suitable for the therapy. Such an indication can be made in writing by, for instance, a handwritten prescription or a computerized report making the corresponding prescription or recommendation.

A “normal cell corresponding to the tumor tissue type” refers to a normal cell from a same tissue type as the tumor tissue. A non-limiting examples is a normal lung cell from a patient having lung tumor, or a normal colon cell from a patient having colon tumor.

The term “amplification” or “amplify” as used herein means one or more methods known in the art for copying a target nucleic acid, thereby increasing the number of copies of a selected nucleic acid sequence. Amplification can be exponential or linear. A target nucleic acid can be either DNA or RNA. The sequences amplified in this manner form an “amplicon.” While the exemplary methods described hereinafter relate to amplification using the polymerase chain reaction (“PCR”), numerous other methods are known in the art for amplification of nucleic acids (e.g., isothermal methods, rolling circle methods, etc.). The skilled artisan will understand that these other methods can be used either in place of, or together with, PCR methods.

The term “complement” as used herein means the complementary sequence to a nucleic acid according to standard Watson/Crick base pairing rules. A complement sequence can also be a sequence of RNA complementary to the DNA sequence or its complement sequence, and can also be a cDNA. The term “substantially complementary” as used herein means that two sequences hybridize under stringent hybridization conditions. The skilled artisan will understand that substantially complementary sequences need not hybridize along their entire length. In particular, substantially complementary sequences comprise a contiguous sequence of bases that do not hybridize to a target or marker sequence, positioned 3′ or 5′ to a contiguous sequence of bases that hybridize under stringent hybridization conditions to a target or marker sequence.

As used herein, the term “hybridize” or “specifically hybridize” refers to a process where two complementary nucleic acid strands anneal to each other under appropriately stringent conditions. Hybridizations are typically conducted with probe-length nucleic acid molecules. Nucleic acid hybridization techniques are well known in the art. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N. Y.; Ausubel, F. M. et al. 1994, Current Protocols in Molecular Biology. John Wiley & Sons, Secaucus, N.J.

“Primer” as used herein refers to an oligonucleotide that is capable of acting as a point of initiation of synthesis when placed under conditions in which primer extension is initiated (e.g., primer extension associated with an application such as PCR). The primer is complementary to a target nucleotide sequence and it hybridizes to a substantially complementary sequence in the target and leads to addition of nucleotides to the 3′-end of the primer in the presence of a DNA or RNA polymerase. The 3′-nucleotide of the primer should generally be complementary to the target sequence at a corresponding nucleotide position for optimal expression and amplification. An oligonucleotide “primer” can occur naturally, as in a purified restriction digest or can be produced synthetically. The term “primer” as used herein includes all forms of primers that can be synthesized including, peptide nucleic acid primers, locked nucleic acid primers, phosphorothioate modified primers, labeled primers, and the like.

Primers are typically between about 5 and about 100 nucleotides in length, such as between about 15 and about 60 nucleotides in length, such as between about 20 and about 50 nucleotides in length, such as between about 25 and about 40 nucleotides in length. In some embodiments, primers can be at least 8, at least 12, at least 16, at least 20, at least 25, at least 30, at least 35, at least 40, at least 45, at least 50, at least 55, at least 60 nucleotides in length. An optimal length for a particular primer application can be readily determined in the manner described in H. Erlich, PCR Technology. Principles and Application for DNA Amplification (1989).

“Probe” as used herein refers to nucleic acid that interacts with a target nucleic acid via hybridization. A probe can be fully complementary to a target nucleic acid sequence or partially complementary. The level of complementarity will depend on many factors based, in general, on the function of the probe. A probe or probes can be used, for example to detect the presence or absence of a mutation in a nucleic acid sequence by virtue of the sequence characteristics of the target. Probes can be labeled or unlabeled, or modified in any of a number of ways well known in the art. A probe can specifically hybridize to a target nucleic acid.

Probes can be DNA, RNA or a RNA/DNA hybrid. Probes can be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes can comprise modified nucleobases, modified sugar moieties, and modified internucleotide linkages. A probe can be fully complementary to a target nucleic acid sequence or partially complementary. A probe can be used to detect the presence or absence of a target nucleic acid. Probes are typically at least about 10, 15, 21, 25, 30, 35, 40, 50, 60, 75, 100 nucleotides or more in length.

“Detecting” as used herein refers to determining the presence of a nucleic acid of interest in a sample or the presence of a protein of interest in a sample. Detection does not require the method to provide 100% sensitivity and/or 100% specificity.

“Detectable label” as used herein refers to a molecule or a compound or a group of molecules or a group of compounds used to identify a nucleic acid or protein of interest. In some cases, the detectable label can be detected directly. In other cases, the detectable label can be a part of a binding pair, which can then be subsequently detected. Signals from the detectable label can be detected by various means and will depend on the nature of the detectable label. Detectable labels can be isotopes, fluorescent moieties, colored substances, and the like. Examples of means to detect detectable label include but are not limited to spectroscopic, photochemical, biochemical, immunochemical, electromagnetic, radiochemical, or chemical means, such as fluorescence, chemifluorescence, or chemiluminescence, or any other appropriate means.

“TaqMan® PCR detection system” as used herein refers to a method for real time PCR. In this method, a TaqMan® probe which hybridizes to the nucleic acid segment amplified is included in the PCR reaction mix. The TaqMan® probe comprises a donor and a quencher fluorophore on either end of the probe and in close enough proximity to each other so that the fluorescence of the donor is taken up by the quencher. However, when the probe hybridizes to the amplified segment, the 5′-exonuclease activity of the Taq polymerase cleaves the probe thereby allowing the donor fluorophore to emit fluorescence which can be detected.

As used herein, the term “sample” or “test sample” refers to any liquid or solid material containing nucleic acids. In suitable embodiments, a test sample is obtained from a biological source (i.e., a “biological sample”), such as cells in culture or a tissue sample from an animal, preferably, a human. In an exemplary embodiment, the sample is a tumor or liquid biopsy sample.

“Target nucleic acid” as used herein refers to segments of a chromosome, a complete gene with or without intergenic sequence, segments or portions a gene with or without intergenic sequence, or sequence of nucleic acids to which probes or primers are designed. Target nucleic acids can include wild type sequences, nucleic acid sequences containing mutations, deletions or duplications, tandem repeat regions, a gene of interest, a region of a gene of interest or any upstream or downstream region thereof. Target nucleic acids can represent alternative sequences or alleles of a particular gene. Target nucleic acids can be derived from genomic DNA, cDNA, or RNA. As used herein, target nucleic acid can be native DNA or a PCR-amplified product.

As used herein the term “stringency” is used in reference to the conditions of temperature, ionic strength, and the presence of other compounds, under which nucleic acid hybridizations are conducted. With high stringency conditions, nucleic acid base pairing will occur only between nucleic acids that have sufficiently long segments with a high frequency of complementary base sequences. Exemplary hybridization conditions are as follows. High stringency generally refers to conditions that permit hybridization of only those nucleic acid sequences that form stable hybrids in 0.018 M NaCl at 65° C. High stringency conditions can be provided, for example, by hybridization in 50% formamide, 5×Denhardt's solution, 5×SSC (saline sodium citrate) 0.2% SDS (sodium dodecyl sulfate) at 42° C., followed by washing in 0.1×SSC, and 0.1% SDS at 65° C. Moderate stringency refers to conditions equivalent to hybridization in 50% formamide, 5×Denhardt's solution, 5×SSC, 0.2% SDS at 42° C., followed by washing in 0.2×SSC, 0.2% SDS, at 65° C. Low stringency refers to conditions equivalent to hybridization in 10% formamide, 5×Denhardt's solution, 6×SSC, 0.2% SDS, followed by washing in 1° SSC, 0.2% SDS, at 50° C.

As used herein the term “substantially identical” refers to a polypeptide or nucleic acid exhibiting at least 50%, 75%, 85%, 90%, 95%, or even 99% identity to a reference amino acid or nucleic acid sequence over the region of comparison. For polypeptides, the length of comparison sequences will generally be at least 20, 30, 40, or 50 amino acids or more, or the full length of the polypeptide. For nucleic acids, the length of comparison sequences will generally be at least 10, 15, 20, 25, 30, 40, 50, 75, or 100 nucleotides or more, or the full length of the nucleic acid.

“TP53 gene” or “tumor protein P53 gene” is a gene that provides instructions for making the tumor suppressor protein p53. The protein p53 plays a role in regulating cell division by preventing cells from growing or proliferating too fast. P53 attaches directly to DNA when DNA damage is detected, where p53 determines whether the DNA will be repaired or whether the cell with undergo apoptosis. If the cell can be repaired, p53 activates DNA repair genes to fix the damage. P53 is crucial in preventing the development of tumors. Mutations in the TP53 gene are universal across cancer types. TP53 mutations are correlated to the onset of various cancers, including but not limited to, breast cancer, bladder cancer, cholangiocarcinoma, lung cancer, melanoma and ovarian cancer.

“EGFR gene” or “epidermal growth factor receptor gene” is a gene that encodes the EGFR protein. EGFR is protein kinase a transmembrane glycoprotein. Mutations in the EGFR gene have been correlated with many types of cancer, including but not limited to non-small cell lung cancer, glioblastoma, and basal-like breast cancers. Tyrosine kinase inhibitors have shown efficacy in EGFR amplified tumors. Thus, TK inhibitors can be an aggressive therapy for the cancers having less favorable prognosis with EGFR as the marker for treatment.

“BRAF gene” or “B-Raf proto-oncogene” is a gene that encodes for an RAF serine/threonine protein kinase. BRAF plays a role in regulating cell division, differentiation and secretion. Mutations in BRAF are often correlated with cancer-causing mutations in melanoma and other forms of cancer as well.

“KIT” gene (also known as c-Kit) encodes a receptor tyrosine kinase. As disclosed by the National Library of Medicine (https://www.ncbi.nlm.nih.gov/gene/3815, last accessed on Dec. 12, 2022), the gene was initially identified as a homolog of the feline sarcoma viral oncogene v-kit and is often referred to as proto-oncogene c-Kit. The canonical form of this glycosylated transmembrane protein has an N-terminal extracellular region with five immunoglobulin-like domains, a transmembrane region, and an intracellular tyrosine kinase domain at the C-terminus. Upon activation by its cytokine ligand, stem cell factor (SCF), this protein phosphorylates multiple intracellular proteins that play a role in in the proliferation, differentiation, migration and apoptosis of many cell types and thereby plays an important role in hematopoiesis, stem cell maintenance, gametogenesis, melanogenesis, and in mast cell development, migration and function. This protein can be a membrane-bound or soluble protein. Mutations in this gene are associated with gastrointestinal stromal tumors, mast cell disease, acute myelogenous leukemia, and piebaldism. Multiple transcript variants encoding different isoforms have been found for this gene. See also, Gene Cards (https://www.genecards.org/cgi-bin/carddisp.pl?gene-KIT, last accessed on Dec. 12, 2022).

The “KMT2C” gene is a member of the myeloid/lymphoid or mixed-lineage leukemia (MLL) family and encodes a nuclear protein with an AT hook DNA-binding domain, a DHHC-type zinc finger, six PHD-type zinc fingers, a SET domain, a post-SET domain and a RING-type zinc finger. This protein is a member of the ASC-2/NCOA6 complex (ASCOM), which possesses histone methylation activity and is involved in transcriptional coactivation. Sequence information for the gene and the encoded protein is found at GeneCards (https://www.genecards.org/cgi-bin/carddisp.pl?gene=KMT2C, last accessed on Dec. 12, 2022).

The “ELF3” gene enables DNA-binding transcription activator activity, RNA polymerase II-specific and sequence-specific double-stranded DNA binding activity. Involved in inflammatory response; negative regulation of transcription, DNA-templated; and positive regulation of transcription by RNA polymerase II. Located in Golgi apparatus; cytosol; and nucleoplasm. Sequence information for the gene and its encoded protein can be found at GeneCards (https://www.genecards.org/cgi-bin/carddisp.pl?gene=ELF3, last accessed on Dec. 12, 2022).

The “APC” gene encodes a tumor suppressor protein that acts as an antagonist of the Wnt signaling pathway. It is also involved in other processes including cell migration and adhesion, transcriptional activation, and apoptosis. Defects in this gene cause familial adenomatous polyposis (FAP), an autosomal dominant pre-malignant disease that usually progresses to malignancy. Mutations in the APC gene have been found to occur in most colorectal cancers, where disease-associated mutations tend to be clustered in a small region designated the mutation cluster region (MCR) and result in a truncated protein product. Sequence information for the gene and the encoded protein is found at GeneCards (https://www.genecards.org/cgi-bin/carddisp.pl?gene=APC, last accessed on Dec. 12, 2022).

The “AIRD1A” gene encodes a member of the SWI/SNF family, whose members have helicase and ATPase activities and are thought to regulate transcription of certain genes by altering the chromatin structure around those genes. The encoded protein is part of the large ATP-dependent chromatin remodeling complex SNF/SWI, which is required for transcriptional activation of genes normally repressed by chromatin. It possesses at least two conserved domains that could be important for its function. Two transcript variants encoding different isoforms have been found for this gene. Sequence information for the gene and the encoded proteins can be found at GeneCards (https://www.genecards.org/cgi-bin/carddisp.pl?gene=ARID1A, last accessed on Dec. 12, 2022).

A “composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “vector” refers to a nucleic acid construct deigned for transfer between different hosts, including but not limited to a plasmid, a virus, a cosmid, a phage, a BAC, a YAC, etc. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. It is to be understood that the vectors contain the necessary regulatory elements for replication or expression of the inserted polynucleotide, including for example promoters or enhancer elements.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

As used herein, the term “isolated cell” generally refers to a cell that is substantially separated from other cells of a tissue. “Immune cells” includes, e.g., white blood cells (leukocytes) which are derived from hematopoietic stem cells (HSC) produced in the bone marrow, lymphocytes (T cells, B cells, natural killer (NK) cells) and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses.

The term “transduce” or “transduction” as it is applied to the production of chimeric antigen receptor cells refers to the process whereby a foreign nucleotide sequence is introduced into a cell. In some embodiments, this transduction is done via a vector.

As used herein, the term “autologous,” in reference to cells refers to cells that are isolated and infused back into the same subject (recipient or host). “Allogeneic” refers to non-autologous cells.

An “effective amount” or “efficacious amount” refers to the amount of an agent, or combined amounts of two or more agents, that, when administered for the treatment of a mammal or other subject, is sufficient to effect such treatment for the disease. The “effective amount” will vary depending on the agent(s), the disease and its severity and the age, weight, etc., of the subject to be treated.

A “solid tumor” is an abnormal mass of tissue that usually does not contain cysts or liquid areas. Solid tumors can be benign or malignant. Different types of solid tumors are named for the type of cells that form them. Examples of solid tumors include sarcomas, carcinomas, and lymphomas.

As used herein, the term “label” intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6^{th} ed). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

As used herein, the term “immunoconjugate” comprises an antibody or an antibody derivative associated with or linked to a second agent, such as a cytotoxic agent, a detectable agent, a radioactive agent, a targeting agent, a human antibody, a humanized antibody, a chimeric antibody, a synthetic antibody, a semisynthetic antibody, or a multispecific antibody.

“Immune response” broadly refers to the antigen-specific responses of lymphocytes to foreign substances. The terms “immunogen” and “immunogenic” refer to molecules with the capacity to elicit an immune response. All immunogens are antigens, however, not all antigens are immunogenic. An immune response disclosed herein can be humoral (via antibody activity) or cell-mediated (via T cell activation). The response may occur in vivo or in vitro. The skilled artisan will understand that a variety of macromolecules, including proteins, nucleic acids, fatty acids, lipids, lipopolysaccharides and polysaccharides have the potential to be immunogenic. The skilled artisan will further understand that nucleic acids encoding a molecule capable of eliciting an immune response necessarily encode an immunogen. The artisan will further understand that immunogens are not limited to full-length molecules, but may include partial molecules.

A host cell can be a eukaryotic or a prokaryotic cell. “Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²p, ³⁵S or ¹²⁵I.

As used herein, the term “purification label” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly (NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein). Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical”, percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, infusion, injection, and topical application. As understood by those of skill in the art, the therapies can be co-administered with other therapies, such as immuno-oncology or chemotherapy. The therapies can be administered simultaneously or concurrently.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

In one aspect, the term “equivalent” or “biological equivalent” of an antibody means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

A “normal cell corresponding to the tumor tissue type” refers to a normal cell from a same tissue type as the tumor tissue. A non-limiting example is a normal lung cell from a patient having lung tumor, or a normal colon cell from a patient having colon tumor.

The term “isolated” as used herein refers to molecules or biologicals or cellular materials being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, the term “monoclonal antibody” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

### Modes for Carrying Out the Disclosure

### Therapeutic Methods

In one aspect, a method of treating inhibiting the growth of a cancer cell or treating a cancer in a subject in need thereof, wherein the subject has a clustered mutation in one or more, or alternatively two or more of, or alternatively three or more of, or alternatively four or more of, or alternatively five or more of, or alternatively six or more of, or alternatively all seven of TP53, EGFR, KIT, KMT2C, ELF3, APC and ARID1A gene(s) or lacks a clustered mutation in a BRAF gene and/or no clustered mutation in the BRAF gene in a sample isolated from the subject is disclosed. The method comprises, consists of, or consists essentially of administering an aggressive therapy to the subject, thereby inhibiting the growth of the cancer cell or treating the cancer in the subject.

The cancer cell can be an animal or a mammalian cell. Non-limiting examples of mammalian cells include human cells, non-human primate cells (e.g., apes, gibbons, chimpanzees, orangutans, monkeys, macaques, and the like), domestic animals (e.g., dogs and cats), farm animals (e.g., horses, cows, goats, sheep, pigs) and experimental animal cells (e.g., mouse, rat, rabbit, guinea pig). In some embodiments, the cell is a human cell. A mammal can be any age or at any stage of development (e.g., an adult, teen, child, infant, or a mammal in utero). A mammal can be male or female. In some embodiments, a subject is a human. In some embodiments, a subject has or is diagnosed of having or is suspected of having a cancer.

The subject can be any animal, typically a mammal. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has or is diagnosed of having or is suspected of having a cancer.

In further aspects, the cancer cell or cancer is selected from a carcinoma, a sarcoma or a blood cancer. In yet further aspects, the cancer cell or cancer is selected from circulatory system, for example, heart (sarcoma [angiosarcoma, fibrosarcoma, rhabdomyosarcoma, liposarcoma], myxoma, rhabdomyoma, fibroma, lipoma and teratoma), mediastinum and pleura, and other intrathoracic organs, vascular tumors and tumor-associated vascular tissue; respiratory tract, for example, nasal cavity and middle ear, accessory sinuses, larynx, trachea, bronchus and lung such as small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal system, for example, esophagus (squamous cell carcinoma, adenocarcinoma, leiomyosarcoma, lymphoma), colon cancer, colorectal cancer, rectal cancer, stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); gastrointestinal stromal tumors and neuroendocrine tumors arising at any site; genitourinary tract, for example, kidney (adenocarcinoma, Wilm's tumor [nephroblastoma], lymphoma, leukemia), bladder and/or urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver, for example, hepatoma (hepatocellular carcinoma), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma); bone, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); reproductive system, for example, gynecological, uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma) and other sites associated with female genital organs; placenta, penis, prostate, testis, and other sites associated with male genital organs; hematologic system, for example, blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; oral cavity, for example, lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx; skin, for example, malignant melanoma, cutaneous melanoma, basal cell carcinoma, squamous cell carcinoma, Karposi's sarcoma, moles dysplastic nevi, lipoma, angioma, dermatofibroma, and keloids; and other tissues comprising connective and soft tissue, retroperitoneum and peritoneum, eye, intraocular melanoma, and adnexa, breast, head or/and neck, anal region, thyroid, parathyroid, adrenal gland and other endocrine glands and related structures, secondary and unspecified malignant neoplasm of lymph nodes, secondary malignant neoplasm of respiratory and digestive systems and secondary malignant neoplasm of other sites.

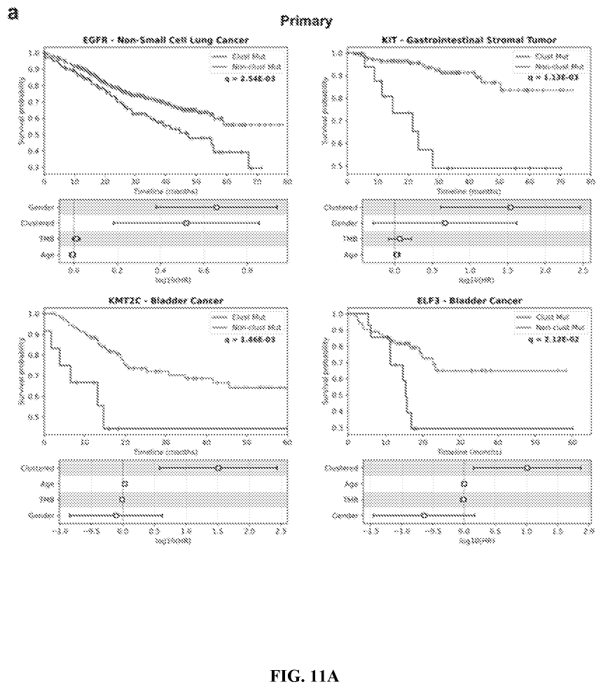

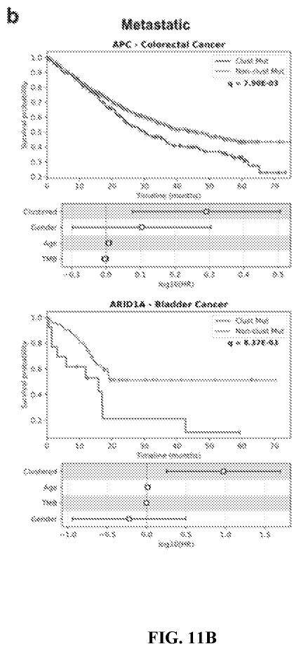

Further, the cancer may be a primary cancer or a metastatic cancer. Moreover, the sample may be a cancer cell isolated from a tumor, a peripheral blood sample or a liquid biopsy. In a further aspect the clustered mutation or lack of the clustered mutation in the BRAF gene is specifically linked to specific cancer type, whether primary or metastatic, see, for example FIGS. 11A and 11B.

The aggressive therapy can be selected from adoptive cell therapy, immune checkpoint blockades including PD1, PD-L1, and CTLA4, pretargeted radioimmunotherapy, oncolytic viral therapy, or cancer vaccines. It also can include TK inhibitors or combination chemotherapy (i.e., two or more agents administered in combination). The particular therapy will depend on the patient, the cancer and the cluster status of the subject.

In a yet further aspect, the aggressive chemotherapy comprises one or more selected from monoclonal antibodies, optionally selected from monospecific antibodies, bispecific antibodies, multispecific antibodies and a bispecific immune cell engager, antibody-drug conjugates, CAR therapies optionally selected from a CAR NK therapy, a CAR T therapy, a CAR cytotoxic T therapy, a CAR gamma-delta T therapy, a CAR NK therapy, cell therapies, inhibitors or antagonists of an inhibitory immune checkpoint, activators or agonists of a stimulatory immune checkpoint optionally selected from an activating ligand, immune regulators, cancer vaccines, and a vector delivering each thereof to a subject optionally in an oncolytic virus therapy.

In another aspect, the aggressive chemotherapy comprises a checkpoint inhibitor. Non-limiting examples of such include GS4224, AMP-224, CA-327, CA-170, BMS-1001, BMS-1166, peptide-57, M7824, MGD013, CX-072, UNP-12, NP-12, or a combination of two or more thereof.

Additional checkpoint inhibitors comprises one or more selected from an anti-PD-1 agent, an anti-PD-L1 agent, an anti-CTLA-4 agent, an anti-LAG-3 agent, an anti-TIM-3 agent, an anti-TIGIT agent, an anti-VISTA agent, an anti-B7-H3 agent, an anti-BTLA agent, an anti-ICOS agent, an anti-GITR agent, an anti-4-1BB agent, an anti-OX40 agent, an anti-CD27 agent, an anti-CD28 agent, an anti-CD40 agent, and an anti-Siglec-15 agent. In a further aspect, the checkpoint inhibitor comprises an anti-PD1 agent or an anti-PD-L1 agent. In one aspect, the anti-PD1 agent comprises an anti-PD1 antibody or an antigen binding fragment thereof. In a further aspect, the anti-PD1 antibody comprises nivolumab, pembrolizumab, cemiplimab, spartalizumab, camrelizumab, sintilimab, tislelizumab, toripalimab, AMF 514, or a combination of two or more thereof. In another aspect, the anti-PD-L1 agent comprises an anti-PD-L1 antibody or an antigen binding fragment thereof. In a further aspect, the anti-PD-L1 antibody comprises avelumab, durvalumab, atezolizumab, envafolimab, or a combination of two or more thereof.

In one aspect, the checkpoint inhibitor comprises an anti-CTLA-4 agent. In another aspect, the anti-CTLA-4 agent comprises an anti-CTLA-4 antibody or an antigen binding fragment thereof. In a yet further aspect, the anti-CTLA-4 antibody comprises ipilimumab, tremelimumab, zalifrelimab, or AGEN1181, or a combination thereof.

In one aspect the therapy further comprises surgical resection of the cancer, tumor or cancer cells. The therapy can be a first-line, second-line, third-line, fourth-line, fifth-line therapy.

### Prognostic and Diagnostic Methods

In yet another aspect, a method for selecting a cancer patient for an aggressive therapy is disclosed. The method comprises, consists of, or consists essentially of assaying for and/or detecting at least one clustered mutation in a gene selected from one or more, or alternatively two or more of, or alternatively three or more of, or alternatively four or more of, or alternatively five or more of, or alternatively six or more of, or alternatively all seven of TP53, EGFR, KIT, KMT2C, ELF3, APC and ARID1A gene(s) or lacks a clustered mutation in a BRAF gene in a sample isolated from the subject wherein the subject is selected for the therapy if the clustered mutation is detected in the sample isolated from the cancer patient or if the BRAF gene is not detected. Non-limiting examples of aggressive therapies are disclosed herein and incorporated herein by reference.

For example, the aggressive therapy can be selected from adoptive cell therapy, immune checkpoint blockades including PD1, PD-L1, and CTLA4, pretargeted radioimmunotherapy, oncolytic viral therapy, or cancer vaccines. It also can include TK inhibitors or combination chemotherapy (i.e., two or more agents administered in combination). The particular therapy will depend on the patient, the cancer and the cluster status of the subject.

In a yet further aspect, the aggressive chemotherapy comprises one or more selected from monoclonal antibodies, optionally selected from monospecific antibodies, bispecific antibodies, multispecific antibodies and a bispecific immune cell engager, antibody-drug conjugates, CAR therapies optionally selected from a CAR NK therapy, a CAR T therapy, a CAR cytotoxic T therapy, a CAR gamma-delta T therapy, a CAR NK therapy, cell therapies, inhibitors or antagonists of an inhibitory immune checkpoint, activators or agonists of a stimulatory immune checkpoint optionally selected from an activating ligand, immune regulators, cancer vaccines, and a vector delivering each thereof to a subject optionally in an oncolytic virus therapy.

In another aspect, the aggressive chemotherapy comprises a checkpoint inhibitor. Non-limiting examples of such include GS4224, AMP-224, CA-327, CA-170, BMS-1001, BMS-1166, peptide-57, M7824, MGD013, CX-072, UNP-12, NP-12, or a combination of two or more thereof.

Additional checkpoint inhibitors comprises one or more selected from an anti-PD-1 agent, an anti-PD-L1 agent, an anti-CTLA-4 agent, an anti-LAG-3 agent, an anti-TIM-3 agent, an anti-TIGIT agent, an anti-VISTA agent, an anti-B7-H3 agent, an anti-BTLA agent, an anti-ICOS agent, an anti-GITR agent, an anti-4-1BB agent, an anti-OX40 agent, an anti-CD27 agent, an anti-CD28 agent, an anti-CD40 agent, and an anti-Siglec-15 agent. In a further aspect, the checkpoint inhibitor comprises an anti-PD1 agent or an anti-PD-L1 agent. In one aspect, the anti-PD1 agent comprises an anti-PD1 antibody or an antigen binding fragment thereof. In a further aspect, the anti-PD1 antibody comprises nivolumab, pembrolizumab, cemiplimab, spartalizumab, camrelizumab, sintilimab, tislelizumab, toripalimab, AMF 514, or a combination of two or more thereof. In another aspect, the anti-PD-L1 agent comprises an anti-PD-L1 antibody or an antigen binding fragment thereof. In a further aspect, the anti-PD-L1 antibody comprises avelumab, durvalumab, atezolizumab, envafolimab, or a combination of two or more thereof. In one aspect, the checkpoint inhibitor comprises an anti-CTLA-4 agent. In another aspect, the anti-CTLA-4 agent comprises an anti-CTLA-4 antibody or an antigen binding fragment thereof. In a yet further aspect, the anti-CTLA-4 antibody comprises ipilimumab, tremelimumab, zalifrelimab, or AGEN1181, or a combination thereof. In yet another aspect, a method for identifying whether a cancer patient is likely to experience a relatively longer or shorter overall survival is disclosed. The method comprises, consists of, or consists essentially of assaying for and/or detecting at least one or more, or alternatively two or more of, or alternatively three or more of, or alternatively four or more of, or alternatively five or more of, or alternatively six or more of, or alternatively all seven of TP53, EGFR, KIT, KMT2C, ELF3, APC and ARID1A gene(s) or lacks a clustered mutation in a BRAF gene in a sample isolated from the patient, wherein the patient is likely to experience longer overall survival if the clustered mutation is detected in BRAF and the patient is likely to experience shorter overall survival if the clustered mutation is detected in at least one or more, or alternatively two or more of, or alternatively three or more of, or alternatively four or more of, or alternatively five or more of, or alternatively six or more of, or alternatively all seven of TP53, EGFR, KIT, KMT2C, ELF3, APC and ARID1A gene(s).

The subject can be any animal, typically a mammal. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has or is diagnosed of having or is suspected of having a cancer.

In further aspects, the cancer cell or cancer is selected from a carcinoma, a sarcoma or a blood cancer. In yet further aspects, the cancer cell or cancer is selected from circulatory system, for example, heart (sarcoma [angiosarcoma, fibrosarcoma, rhabdomyosarcoma, liposarcoma], myxoma, rhabdomyoma, fibroma, lipoma and teratoma), mediastinum and pleura, and other intrathoracic organs, vascular tumors and tumor-associated vascular tissue; respiratory tract, for example, nasal cavity and middle ear, accessory sinuses, larynx, trachea, bronchus and lung such as small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal system, for example, esophagus (squamous cell carcinoma, adenocarcinoma, leiomyosarcoma, lymphoma), stomach (carcinoma, lymphoma, leiomyosarcoma), colon cancer, colorectal cancer, rectal cancer, gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); gastrointestinal stromal tumors and neuroendocrine tumors arising at any site; genitourinary tract, for example, kidney (adenocarcinoma, Wilm's tumor [nephroblastoma], lymphoma, leukemia), bladder and/or urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver, for example, hepatoma (hepatocellular carcinoma), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma); bone, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); reproductive system, for example, gynecological, uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma) and other sites associated with female genital organs; placenta, penis, prostate, testis, and other sites associated with male genital organs; hematologic system, for example, blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; oral cavity, for example, lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx; skin, for example, malignant melanoma, cutaneous melanoma, basal cell carcinoma, squamous cell carcinoma, Karposi's sarcoma, moles dysplastic nevi, lipoma, angioma, dermatofibroma, and keloids; and other tissues comprising connective and soft tissue, retroperitoneum and peritoneum, eye, intraocular melanoma, and adnexa, breast, head or/and neck, anal region, thyroid, parathyroid, adrenal gland and other endocrine glands and related structures, secondary and unspecified malignant neoplasm of lymph nodes, secondary malignant neoplasm of respiratory and digestive systems and secondary malignant neoplasm of other sites.

The cancer can be primary or metatstatic. In some aspects, the clustered mutation is specifically linked to a primary or metastatic cancer, see, e.g., FIGS. 11A and 11B.

Any suitable method for identifying the genotype in the patient sample can be used and the disclosures described herein are not to be limited to these methods. For the purpose of illustration only, the genotype is determined by a method comprising, or alternatively consisting essentially of, or yet further consisting of, sequencing, hybridization, nucleic acid amplification, including polymerase chain reaction (PCR), real-time PCR, reverse transcriptase PCR (RT-PCR), nested PCR, ligase chain reaction, or PCR-RFLP, or microarray. These methods as well as equivalents or alternatives thereto are described herein.

Information obtained using the diagnostic assays described herein is useful for determining if a subject will likely, more likely, or less likely to respond to cancer treatment of a given type. Based on the prognostic information, a doctor can recommend a therapeutic protocol, useful for treating reducing the malignant mass or tumor in the patient or treat cancer in the individual.

In addition, knowledge of the identity of a particular allele in an individual (the gene profile) allows customization of therapy for a particular disease to the individual's genetic profile, the goal of “pharmacogenomics”. For example, an individual's genetic profile can enable a doctor: 1) to more effectively prescribe a drug that will address the molecular basis of the disease or condition; 2) to better determine the appropriate dosage of a particular drug and 3) to identify novel targets for drug development. The identity of the genotype or expression patterns of individual patients can then be compared to the genotype or expression profile of the disease to determine the appropriate drug and dose to administer to the patient.

The ability to target populations expected to show the highest clinical benefit, based on the normal or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling.

### Biological Sample Collection and Preparation

The methods and compositions disclosed herein can be used to detect nucleic acids associated with the genetic polymorphisms identified herein using a biological sample obtained from a patient. Biological samples can be obtained by standard procedures and can be used immediately or stored, under conditions appropriate for the type of biological sample, for later use. Any liquid or solid biological material obtained from the patient believed to contain nucleic acids comprising the region the polymorphic region can be a suitable sample. The sample can be a tumor sample, a peripheral blood sample or a liquid biopsy.

Methods of obtaining test samples are known to those of skill in the art and include, but are not limited to, aspirations, tissue sections, swabs, drawing of blood or other fluids, surgical or needle biopsies.

In some aspects, the biological sample is a tissue or a cell sample. Suitable patient samples in the methods include, but are not limited to, blood, plasma, serum, a biopsy tissue, fine needle biopsy sample, amniotic fluid, plasma, pleural fluid, saliva, semen, serum, tissue or tissue homogenates, frozen or paraffin sections of tissue or combinations thereof. In some aspects, the biological sample comprises, or alternatively consisting essentially of, or yet further consisting of, at least one of a tumor cell, a normal cell adjacent to a tumor, a normal cell corresponding to the tumor tissue type, a blood cell, a peripheral blood lymphocyte, or combinations thereof. In some aspects, the biological sample is an original sample recently isolated from the patient, a fixed tissue, a frozen tissue, a resection tissue, or a microdissected tissue. In some aspects, the biological samples are processed, such as by sectioning of tissues, fractionation, purification, nucleic acid isolation, or cellular organelle separation.

In some embodiments, nucleic acid (DNA or RNA) is isolated from the sample according to any methods known to those of skill in the art. In some aspects, genomic DNA is isolated from the biological sample. In some aspects, RNA is isolated from the biological sample. In some aspects, cDNA is generated from mRNA in the sample. In some embodiments, the nucleic acid is not isolated from the biological sample (e.g., the polymorphism is detected directly from the biological sample).

### Detection of Clustered Mutations and Polymorphisms

Methods to detect or assay for clustered mutations are known in the art and described herein. In some aspects, detection of a clustered mutations or polymorphisms can be accomplished by molecular cloning of the specified allele and subsequent sequencing of that allele using techniques known in the art, in some aspects, after isolation of a suitable nucleic acid sample. In some aspects, the gene sequences can be amplified directly from a genomic DNA preparation from the biological sample using PCR, and the sequence composition is determined by sequencing the amplified product (i.e., amplicon). Alternatively, the PCR product can be analyzed following digestion with a restriction enzyme, a method known as PCR-RFLP.

In some embodiments, the clustered mutations or polymorphism is detected using allele specific hybridization using probes overlapping the polymorphic site. In some aspects, the nucleic acid probes are between 5 and 40 nucleotides in length. In some aspects, the nucleic acid probes are about 5, about 10, about 15, about 20, about 25, about 30, about 35, or about 40 or more nucleotides flanking the polymorphic site.

In another embodiment of the disclosure, several nucleic acid probes capable of hybridizing specifically to the nucleic acid containing the allelic variant are attached to a solid phase support, e.g., a “chip” or “microarray. Such gene chips or microarrays can be used to detect genetic variations by a number of techniques known to one of skill in the art. In one technique, oligonucleotides are arrayed on a gene chip for determining the DNA sequence by the sequencing by hybridization approach. The probes of the disclosure also can be used for fluorescent detection of a genetic sequence. A probe also can be affixed to an electrode surface for the electrochemical detection of nucleic acid sequences.

In one aspect, “gene chips” or “microarrays” containing probes or primers for the gene of interest are provided alone or in combination with other probes and/or primers. A suitable sample is obtained from the patient extraction of genomic DNA, RNA, or any combination thereof and amplified if necessary. The DNA or RNA sample is contacted to the gene chip or microarray panel under conditions suitable for hybridization of the gene(s) of interest to the probe(s) or primer(s) contained on the gene chip or microarray. The probes or primers can be detectably labeled thereby identifying the polymorphism in the gene(s) of interest. Alternatively, a chemical or biological reaction can be used to identify the probes or primers which hybridized with the DNA or RNA of the gene(s) of interest. The genetic profile of the patient is then determined with the aid of the aforementioned apparatus and methods.

In some aspects, whole genome sequencing, in particular with the “next generation sequencing” techniques, which employ massively parallel sequencing of DNA templates, can be used to obtain genotypes of relevant polymorphisms. Exemplary NGS sequencing platforms for the generation of nucleic acid sequence data include, but are not limited to, Illumina's sequencing by synthesis technology (e.g., Illumina MiSeq or HiSeq System), Life Technologies' Ion Torrent semiconductor sequencing technology (e.g., Ion Torrent PGM or Proton system), the Roche (454 Life Sciences) GS series and Qiagen (Intelligent BioSystems) Gene Reader sequencing platforms.

In some aspects, nucleic acid comprising, or alternatively consisting essentially of, or yet further consisting of the polymorphism is amplified to produce an amplicon containing the polymorphism. Nucleic acids can be amplified by various methods known to the skilled artisan. Nucleic acid amplification can be linear or exponential. Amplification is generally carried out using polymerase chain reaction (PCR) technologies. Alternative or modified PCR amplification methods can also be used and include, for example, isothermal amplification methods, rolling circle methods, Hot-start PCR, real-time PCR, Allele-specific PCR, Assembly PCR or Polymerase Cycling Assembly (PCA), Asymmetric PCR, Colony PCR, Emulsion PCR, Fast PCR, Real-Time PCR, nucleic acid ligation, Gap Ligation Chain Reaction (Gap LCR), Ligation-mediated PCR, Multiplex Ligation-dependent Probe Amplification, (MLPA), Gap Extension Ligation PCR (GEXL-PCR), quantitative PCR (Q-PCR), Quantitative real-time PCR (QRT-PCR), multiplex PCR, Helicase-dependent amplification, Intersequence-specific (ISSR) PCR, Inverse PCR, Linear-After-The-Exponential-PCR (LATE-PCR), Methylation-specific PCR (MSP), Nested PCR, Overlap-extension PCR, PAN-AC assay, Reverse Transcription PCR (RT-PCR), Rapid Amplification of cDNA Ends (RACE PCR), Single molecule amplification PCR (SMA PCR), Thermal asymmetric interlaced PCR (TAIL-PCR), Touchdown PCR, long PCR, nucleic acid sequencing (including DNA sequencing and RNA sequencing), transcription, reverse transcription, duplication, DNA or RNA ligation, and other nucleic acid extension reactions known in the art. The skilled artisan will understand that other methods can be used either in place of, or together with, PCR methods, including enzymatic replication reactions developed in the future. See, e.g., Saiki, “Amplification of Genomic DNA” in PCR Protocols, Innis et al., eds., Academic Press, San Diego, Calif., 13-20 (1990); Wharam, et al., 29 (11) Nucleic Acids Res, E54-E54 (2001); Hafner, et al., 30 (4) Biotechniques, 852-6, 858, 860 passim (2001).

In some aspects, nucleic acid comprising, or alternatively consisting essentially of, or yet further consisting of the polymorphism of interest is amplified to produce an amplicon. In some aspects, a nucleic acid containing the region of interest is amplified using a forward primer and a reverse primer the flank the region of interest. In some aspects, the amplicon containing the region of interest (e.g. an amplicon having the polymorphic sequence) is detected by hybridizing a nucleic acid probe containing the polymorphism or a complement thereof to the corresponding complementary strand of the amplicon and detecting the hybrid formed between the nucleic acid probe and the complementary strand of the amplicon. In some aspects, amplicon containing the region of interest is sequenced (e.g., dideoxy chain termination methods (Sanger method and variants thereof), Maxam & Gilbert sequencing, pyrosequencing, exonuclease digestion and next-generation sequencing methods).

In some embodiments, the amplification includes a labeled primer or probe, thereby allowing detection of the amplification products corresponding to that primer or probe. In particular embodiments, the amplification can include a multiplicity of labeled primers or probes; such primers can be distinguishably labeled, allowing the simultaneous detection of multiple amplification products.

In some embodiments, the amplification products are detected by any of a number of methods such as gel electrophoresis, column chromatography, hybridization with a nucleic acid probe, or sequencing the amplicon.

Detectable labels can be used to identify the primer or probe hybridized to a genomic nucleic acid or amplicon. Detectable labels include but are not limited to fluorophores, isotopes (e.g., ³²P, ³³P, ³⁵S, ³H, ¹⁴C, ¹²⁵I, ¹³¹I) electron-dense reagents (e.g., gold, silver), nanoparticles, enzymes commonly used in an ELISA (e.g., horseradish peroxidase, beta-galactosidase, luciferase, alkaline phosphatase), chemiluminescent compounds, colorimetric labels (e.g., colloidal gold), magnetic labels (e.g., Dynabeads®), biotin, digoxigenin, haptens, proteins for which antisera or monoclonal antibodies are available, ligands, hormones, oligonucleotides capable of forming a complex with the corresponding oligonucleotide complement.

In one embodiment, a primer or probe is labeled with a fluorophore that emits a detectable signal. The term “fluorophore” as used herein refers to a molecule that absorbs light at a particular wavelength (excitation frequency) and subsequently emits light of a longer wavelength (emission frequency). While a suitable reporter dye is a fluorescent dye, any reporter dye that can be attached to a detection reagent such as an oligonucleotide probe or primer is suitable for use in the methods described. Suitable fluorescent moieties include, but are not limited to, the following fluorophores working individually or in combination: 4-acetamido-4′-isothiocyanatostilbene-2,2′disulfonic acid; acridine and derivatives, e.g. acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl) maleimide; anthranilamide; Black Hole Quencher™ (BHQ™) dyes (biosearch Technologies); BODIPY dyes: BODIPY® R-6G, BODIPY® 530/550, BODIPY® FL; Brilliant Yellow; coumarin and derivatives: coumarin, 7-amino-4-methylcoumarin (AMC, Coumarin 120), 7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescein (TET); fluorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSYR 9; QSYR 21; QSYR 35 (Molecular Probes); Reactive Red 4 (Cibacron® Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; and tetramethyl rhodamine isothiocyanate (TRITC).

In some aspects, the primer or probe is further labeled with a quencher dye such as Tamra, Dabcyl, or Black Hole Quencher® (BHQ), especially when the reagent is used as a self-quenching probe such as a TaqMan® (U.S. Pat. Nos. 5,210,015 and 5,538,848) or Molecular Beacon probe (U.S. Pat. Nos. 5,118,801 and 5,312,728), or other stemless or linear beacon probe (Livak et al., 1995, PCR Method Appl., 4:357-362; Tyagi et al, 1996, Nature Biotechnology, 14:303-308; Nazarenko et al., 1997, Nucl. Acids Res., 25:2516-2521; U.S. Pat. Nos. 5,866,336 and 6,117,635).

In some aspects, methods for real time PCR use fluorescent primers/probes, such as the TaqMan® primers/probes (Heid, et al., Genome Res 6:986-994, 1996), molecular beacons, and Scorpion™ primers/probes. Real-time PCR quantifies the initial amount of the template with more specificity, sensitivity and reproducibility, than other forms of quantitative PCR, which detect the amount of final amplified product. Real-time PCR does not detect the size of the amplicon. The probes employed in Scorpion®™ and TaqMan® technologies are based on the principle of fluorescence quenching and involve a donor fluorophore and a quenching moiety. The term “donor fluorophore” as used herein means a fluorophore that, when in close proximity to a quencher moiety, donates or transfers emission energy to the quencher. As a result of donating energy to the quencher moiety, the donor fluorophore will itself emit less light at a particular emission frequency that it would have in the absence of a closely positioned quencher moiety. The term “quencher moiety” as used herein means a molecule that, in close proximity to a donor fluorophore, takes up emission energy generated by the donor and either dissipates the energy as heat or emits light of a longer wavelength than the emission wavelength of the donor. In the latter case, the quencher is considered to be an acceptor fluorophore. The quenching moiety can act via proximal (i.e., collisional) quenching or by Forster or fluorescence resonance energy transfer (“FRET”). Quenching by FRET is generally used in TaqMan® primers/probes while proximal quenching is used in molecular beacon and Scorpion™ type primers/probes.

The detectable label can be incorporated into, associated with or conjugated to a nucleic acid primer or probe. Labels can be attached by spacer arms of various lengths to reduce potential steric hindrance or impact on other useful or desired properties. See, e.g., Mansfield, Mol. Cell. Probes (1995), 9:145-156.

Detectable labels can be incorporated into nucleic acid probes by covalent or non-covalent means, e.g., by transcription, such as by random-primer labeling using Klenow polymerase, or nick translation, or, amplification, or equivalent as is known in the art. For example, a nucleotide base is conjugated to a detectable moiety, such as a fluorescent dye, e.g., Cy3™ or Cy5™ and then incorporated into nucleic acid probes during nucleic acid synthesis or amplification. Nucleic acid probes can thereby be labeled when synthesized using Cy3™- or Cy5™-dCTP conjugates mixed with unlabeled dCTP.

Nucleic acid probes can be labeled by using PCR or nick translation in the presence of labeled precursor nucleotides, for example, modified nucleotides synthesized by coupling allylamine-dUTP to the succinimidyl-ester derivatives of the fluorescent dyes or haptens (such as biotin or digoxigenin) can be used; this method allows custom preparation of most common fluorescent nucleotides, see, e.g., Henegariu et al., Nat. Biotechnol. (2000), 18:345-348.

Nucleic acid probes can be labeled by non-covalent means known in the art. For example, Kreatech Biotechnology's Universal Linkage System® (ULS®) provides a non-enzymatic labeling technology, wherein a platinum group forms a co-ordinative bond with DNA, RNA or nucleotides by binding to the N7 position of guanosine. This technology can also be used to label proteins by binding to nitrogen and sulfur containing side chains of amino acids. See, e.g., U.S. Pat. Nos. 5,580,990; 5,714,327; and 5,985,566; and European Patent No. 0539466.

Labeling with a detectable label also can include a nucleic acid attached to another biological molecule, such as a nucleic acid, e.g., an oligonucleotide, or a nucleic acid in the form of a stem-loop structure as a “molecular beacon” or an “aptamer beacon”. Molecular beacons as detectable moieties are described; for example, Sokol (Proc. Natl. Acad. Sci. USA (1998), 95:11538-11543) synthesized “molecular beacon” reporter oligodeoxynucleotides with matched fluorescent donor and acceptor chromophores on their 5′ and 3′ ends. In the absence of a complementary nucleic acid strand, the molecular beacon remains in a stem-loop conformation where fluorescence resonance energy transfer prevents signal emission. On hybridization with a complementary sequence, the stem-loop structure opens increasing the physical distance between the donor and acceptor moieties thereby reducing fluorescence resonance energy transfer and allowing a detectable signal to be emitted when the beacon is excited by light of the appropriate wavelength. See also, e.g., Antony (Biochemistry (2001), 40:9387-9395), describing a molecular beacon consist of a G-rich 18-mer triplex forming oligodeoxyribonucleotide. See also U.S. Pat. Nos. 6,277,581 and 6,235,504.

Aptamer beacons are similar to molecular beacons; see, e.g., Hamaguchi, Anal. Biochem. (2001), 294:126-131; Poddar, Mol. Cell. Probes (2001), 15:161-167; Kaboev, Nucleic Acids Res. (2000), 28: E94. Aptamer beacons can adopt two or more conformations, one of which allows ligand binding. A fluorescence-quenching pair is used to report changes in conformation induced by ligand binding. See also, e.g., Yamamoto et al., Genes Cells (2000), 5:389-396; Smimov et al., Biochemistry (2000), 39:1462-1468.

The nucleic acid primer or probe can be indirectly detectably labeled via a peptide. A peptide can be made detectable by incorporating predetermined polypeptide epitopes recognized by a secondary reporter (e.g., leucine zipper pair sequences, binding sites for secondary antibodies, transcriptional activator polypeptide, metal binding domains, epitope tags). A label can also be attached via a second peptide that interacts with the first peptide (e.g., S—association).

As readily recognized by one of skill in the art, detection of the complex containing the nucleic acid from a sample hybridized to a labeled probe can be achieved through use of a labeled antibody against the label of the probe. In one example, the probe is labeled with digoxigenin and is detected with a fluorescent labeled anti-digoxigenin antibody. In another example, the probe is labeled with FITC, and detected with fluorescent labeled anti-FITC antibody. These antibodies are readily available commercially. In another example, the probe is labeled with FITC, and detected with anti-FITC antibody primary antibody and a labeled anti-anti FITC secondary antibody.

Nucleic acids can be amplified prior to detection or can be detected directly during an amplification step (i.e., “real-time” methods, such as in TaqMan® and Scorpion™ methods). In some embodiments, the target sequence is amplified using a labeled primer such that the resulting amplicon is detectably labeled. In some embodiments, the primer is fluorescently labeled. In some embodiments, the target sequence is amplified and the resulting amplicon is detected by electrophoresis.

With regard to the exemplary primers and probes, those skilled in the art will readily recognize that nucleic acid molecules can be double-stranded molecules and that reference to a particular site on one strand refers, as well, to the corresponding site on a complementary strand. In defining a variant position, allele, or nucleotide sequence, reference to an adenine, a thymine (uridine), a cytosine, or a guanine at a particular site on one strand of a nucleic acid molecule also defines the thymine (uridine), adenine, guanine, or cytosine (respectively) at the corresponding site on a complementary strand of the nucleic acid molecule. Thus, reference can be made to either strand in order to refer to a particular variant position, allele, or nucleotide sequence. Probes and primers, can be designed to hybridize to either strand and detection methods disclosed herein can generally target either strand.

In some embodiments, the primers and probes comprise additional nucleotides corresponding to sequences of universal primers (e.g., T7, M13, SP6, T3) which add the additional sequence to the amplicon during amplification to permit further amplification and/or prime the amplicon for sequencing.

As noted above, the disclosure further provides methods of treating a patient selected by any method of the above embodiments, or identified as likely to experience a more favorable clinical outcome by any of the above methods, following the therapy. In some embodiments, the methods entail administering to the patients such a therapy. The therapy can be any one of the group of: a first line, second line, third line, a fourth line, or a fifth line therapy.

### Compositions and Modes of Administration

The agents or drugs can be administered as a composition. A “composition” typically intends a combination of the active agent and another carrier, e.g., compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates.

Various delivery systems are known and can be used to administer a chemotherapeutic agent of the disclosure, e.g., encapsulation in liposomes, microparticles, microcapsules, expression by recombinant cells, receptor-mediated endocytosis. See e.g., Wu and Wu (1987) J. Biol. Chem. 262:4429-4432 for construction of a therapeutic nucleic acid as part of a retroviral or other vector, etc. Methods of delivery include but are not limited to intra-arterial, intra-muscular, intravenous, intranasal and oral routes. In a specific embodiment, it can be desirable to administer the pharmaceutical compositions of the disclosure locally to the area in need of treatment; this can be achieved by, for example, and not by way of limitation, local infusion during surgery, by injection or by means of a catheter.

The agents identified herein as effective for their intended purpose can be administered to subjects or individuals identified by the methods herein as suitable for the therapy. Therapeutic amounts can be empirically determined and will vary with the pathology being treated, the subject being treated and the efficacy and toxicity of the agent.

Methods of administering pharmaceutical compositions are well known to those of ordinary skill in the art and include, but are not limited to, oral, microinjection, intravenous or parenteral administration. The compositions are intended for topical, oral, or local administration as well as intravenously, subcutaneously, or intramuscularly. Administration can be effected continuously or intermittently throughout the course of the treatment. Methods of determining the most effective means and dosage of administration are well known to those of skill in the art and will vary with the cancer being treated and the patient and the subject being treated. Single or multiple administrations can be carried out with the dose level and pattern being selected by the treating physician.

### Kits

Kits or panel for use in detecting the polymorphism of interest in patient biological samples are provided. In some embodiments, a kit comprises, or consists essentially of, or yet further consists of at least one reagent necessary to perform the assay. For example, the kit can comprise an enzyme, a buffer or any other necessary reagent (e.g. PCR reagents and buffers). For example, in some aspects, a kit contains, in an amount sufficient for at least one assay, any of the hybridization assay probes, amplification primers, and/or antibodies suitable for detection in a packaging material.

The various components of the kit can be provided in a variety of forms. For example, in some aspects, the required enzymes, the nucleotide triphosphates, the probes, primers, and/or antibodies are be provided as a lyophilized reagent. These lyophilized reagents can be pre-mixed before lyophilization so that when reconstituted they form a complete mixture with the proper ratio of each of the components ready for use in the assay. In addition, the kits can contain a reconstitution reagent for reconstituting the lyophilized reagents of the kit. In exemplary kits for amplifying target nucleic acid derived from a colorectal cancer patients, the enzymes, nucleotide triphosphates and required cofactors for the enzymes are provided as a single lyophilized reagent that, when reconstituted, forms a proper reagent for use in the present amplification methods.

Typically, the kits will also include instructions recorded in a tangible form (e.g., contained on paper or an electronic medium) for using the packaged probes, primers, and/or antibodies in a detection assay for determining the presence or amount of the polymorphism of interest in a test sample.

In some aspects, the kits further comprise a solid support for anchoring the nucleic acid of interest on the solid support. The target nucleic acid can be anchored to the solid support directly or indirectly through a capture probe anchored to the solid support and capable of hybridizing to the nucleic acid of interest. Examples of such solid support include but are not limited to beads, microparticles (for example, gold and other nano particles), microarray, microwells, multiwell plates. The solid surfaces can comprise a first member of a binding pair and the capture probe or the target nucleic acid can comprise a second member of the binding pair. Binding of the binding pair members will anchor the capture probe or the target nucleic acid to the solid surface. Examples of such binding pairs include but are not limited to biotin/streptavidin, hormone/receptor, ligand/receptor, and antigen/antibody.

In one aspect, the kit further comprises, or consists essentially of, or yet further consists of an effective amount of the therapy.

The kit can comprise at least one probe or primer which is capable of specifically hybridizing to the gene of interest and instructions for use. For example, in some aspects, the kits comprise at least one of the above described nucleic acids. Exemplary kits for amplifying at least a portion of the gene of interest comprise two primers. For example, in some embodiments, the kit comprises, or consists essentially of, or yet further consists of a forward primer and a reverse primer that flank the polymorphism.

In some embodiments, the kit further comprises, or consists essentially of, or yet further consists of a nucleic acid probe for the detection of the amplicon. In some embodiments, the nucleic acid probe has about 5, about 10, about 15, about 20, or about 25, or about 30, about 35, about 40 or more contiguous nucleotides. In some aspects, the nucleic acid primers and/or probes are lyophilized.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the surface is silica or glass. In another embodiment, the surface is a metal electrode.

The test samples used in the diagnostic kits include cells, protein or membrane extracts of cells, or biological fluids such as sputum, blood, serum, plasma, or urine. The test samples can also be a tumor cell, a normal cell adjacent to a tumor, a normal cell corresponding to the tumor tissue type, a blood cell, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kits can include all or some of the positive controls, negative controls, reagents, primers, sequencing markers, probes and antibodies described herein for determining the subject's genotype in the polymorphic region of the gene of interest or target region.

As amenable, these suggested kit components can be packaged in a manner customary for use by those of skill in the art. For example, these suggested kit components can be provided in solution or as a liquid dispersion or the like.

Typical packaging materials would include solid matrices such as glass, plastic, paper, foil, micro-particles and the like, capable of holding within fixed limits hybridization assay probes, and/or amplification primers. Thus, for example, the packaging materials can include glass vials used to contain sub-milligram (e.g., picogram or nanogram) quantities of a contemplated probe, primer, or antibodies or they can be microtiter plate wells to which probes, primers, or antibodies have been operatively affixed, i.e., linked so as to be capable of participating in an amplification and/or detection methods.

The instructions will typically indicate the reagents and/or concentrations of reagents and at least one assay method parameter which might be, for example, the relative amounts of reagents to use per amount of sample. In addition, such specifics as maintenance, time periods, temperature, and buffer conditions can also be included.

The diagnostic systems contemplate kits having any of the hybridization assay probes, amplification primers, or antibodies described herein, whether provided individually or in one of the combinations described above, for use in determining the presence or amount of a polymorphism of interest, or as identified herein.

The disclosure now being generally described, it will be more readily understood by reference to the following example which is included merely for purposes of illustration of certain aspects and embodiments of the present disclosure, and are not intended to limit the disclosure.

### Experimental Methods

### Experiment No. 1

### The Landscape of Clustered Mutations

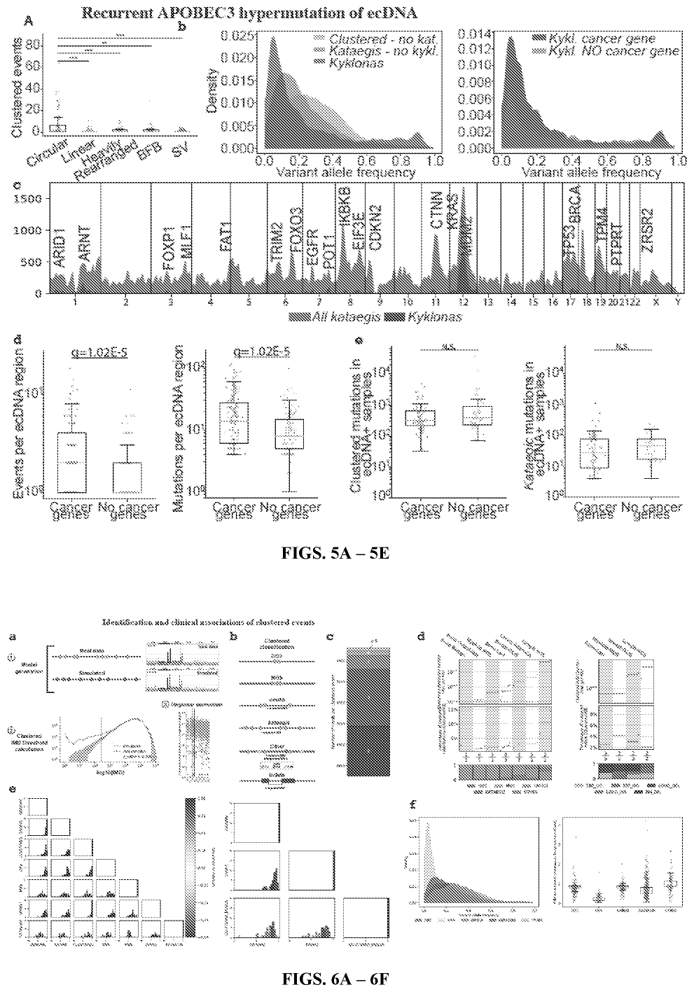

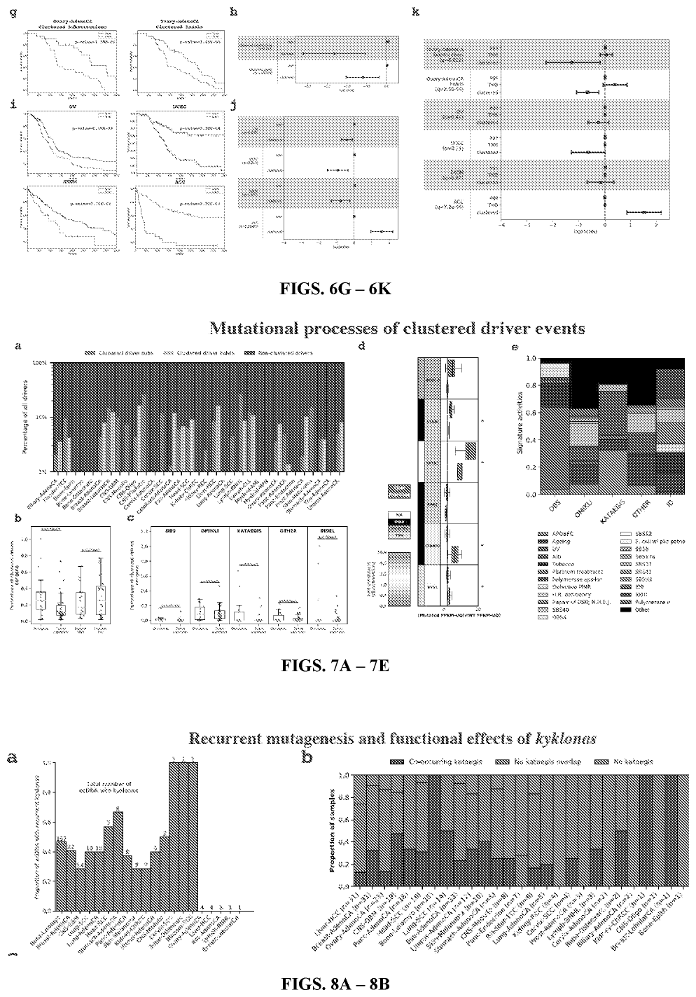

To identify clustered mutations, a sample dependent intra-mutational distance (IMD) cutoff was derived where mutations below the cutoff were unlikely to occur by chance (q-value <0.01). A statistical approach utilizing the IMD cutoff, variant allele frequencies (VAFs), and corrections for local sequence context was applied to each specimen (see FIG. 6A). Clustered mutations with consistent VAFs were subclassified into four categories (see FIG. 6B). Doublet-base substitutions (DBSs) and multi-base substitutions (MBSs) were characterized, respectively, as 2 and ≥3 adjacent mutations (IMD=1). Multiple substitutions each with IMD >1 bp and below the sample-dependent cutoff were characterized as either omikli (2-3 substitutions) or kataegis (>4 substitutions). Clustered substitutions with inconsistent VAFs were classified as other. While clustered indels were not subclassified into different categories, most events resembled diffuse hypermutation with 92.3% of events having only two indels (see FIG. 6C).

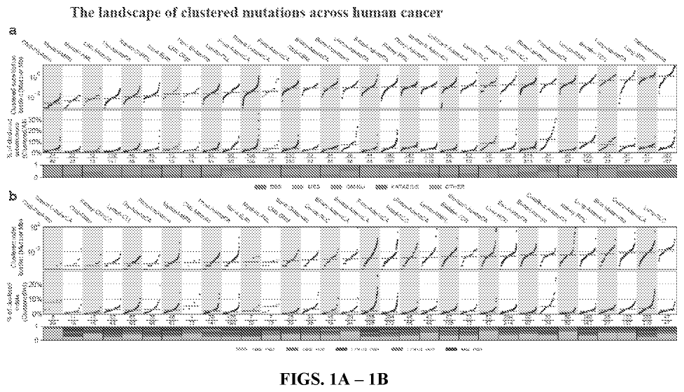

Examining 2,583 whole-genome sequenced cancers from the Pan-Cancer Analysis of Whole Genomes (PCAWG) project revealed a total of 1,686,013 clustered single-base substitutions and 21,368 clustered indels (FIG. 1 and FIG. 6D). DBSs, MBSs, omikli, and kataegis comprise 45.7%, 0.7%, 37.2%, and 7.0% of clustered substitutions across all samples, respectively, with their distributions varying greatly within and across cancer types. For example, melanoma had the highest clustered substitution burden with ultraviolet light associated doublets (viz., CC>TT) accounting for 74.2% of clustered mutations; however, these contributed only 5.3% of all substitutions in melanoma (FIG. 1A). In contrast, 11.5% of all substitutions in bone leiomyosarcomas were clustered with omikli and kataegis constituting 43.8% and 46.7% of these mutations, respectively (FIG. 1A). Clustered indels exhibited similarly diverse patterns within and across cancer types (FIG. 1B). For example, the highest mutational burden of clustered indels was observed in lung and ovarian cancers. Clustered indels in lung cancer accounted for only 2.6% of all indels and were characterized by lbp deletions. In contrast, clustered long indels at microhomologies were commonly found in ovarian and breast cancers and contributed >10% of all indels in a subset of samples (FIG. 1B). Correlations between the total number of mutations and the number of clustered mutations were observed for DBSs and omikli but not for MBSs, kataegis, or indels (FIG. 6E). In most cancers, DBSs and omikli had VAFs consistent with the ones of non-clustered mutations while MBSs and kataegis tended to have lower VAFs (FIG. 6F). Kataegic events contained 4 to 44 mutations with 81% of events being strand-coordinated, indicative of damage or enzymatic changes on a single DNA strand.

The overall survival was compared between patients with cancers harboring high and low numbers of clustered mutations within whole-genome sequenced PCAWG and whole-exome sequenced TCGA cancer types³⁶. Better overall survival was observed only in whole-genome sequenced ovarian cancers containing high-levels of clustered substitutions or clustered indels (q-values <0.05; FIGS. 6G and 6H). Conversely, whole-exome sequenced adrenocortical carcinomas containing clustered substitutions were associated with a worse overall survival (q-value=7.2E-05; FIGS. 6I to 6K).

### Signatures of Clustered Mutations

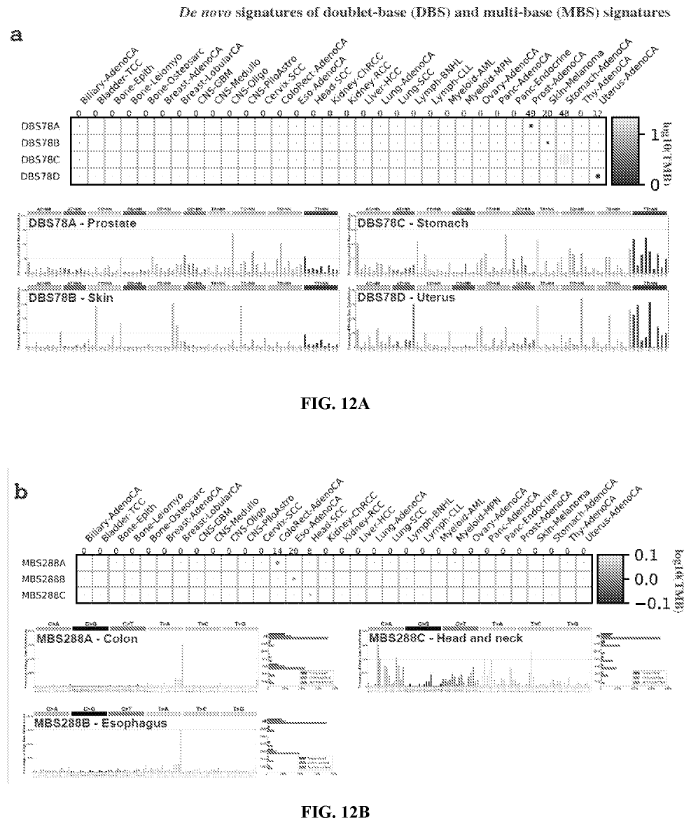

Mutational signature analysis was performed for each category of clustered events elucidating 12 DBS, 5 MBS, 17 omikli, 9 kataegic, and 6 clustered indel signatures (FIG. 2). While DBS signatures have been previously described¹, prior analysis combined DBSs and MBSs into a single class¹. Separating these events into individual classes revealed that a multitude of processes can give rise to DBSs while most MBSs are attributable to signatures associated with tobacco smoking (SBS4) or ultraviolet light (SBS7). Additional DBS and MBS signatures were found within a small subset of cancer types (FIG. 12).

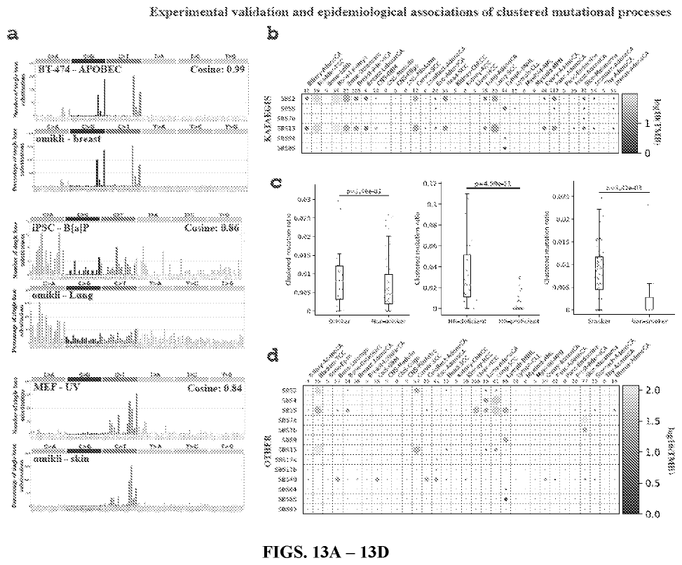

In cancer genomes, omikli were previously attributed to APOBEC3 mutagenesis⁶ with some indirect evidence from experimental models^{23,37,38} Applicant's analysis of sequencing data³⁹ from the clonally expanded breast cancer cell line BT-474 with active APOBEC3 mutagenesis experimentally confirmed the existence of APOBEC3-associated omikli events (cosine similarity: 0.99; FIG. 13A). Only 16.2% of omikli events across the 2,583 cancer genomes matched the APOBEC3 mutational pattern indicating that a plethora of other processes can give rise to diffuse clustered hypermutation. Importantly, Applicant's analysis revealed omikli due to tobacco smoking (SBS4), clock-like mutational processes (SBS5), ultraviolet light (SBS7), both direct and indirect mutations from AID (SBS9 and SBS85), and multiple mutational signatures with unknown etiology in different cancer types (SBS8, SBS12, SBS17a/b, SBS28, SBS40, and SBS41; FIG. 2). Cell lines previously exposed to benzo[a]pyrene⁴⁰ and ultraviolet light⁴¹ confirmed the generation of omikli events due to these two environmental exposures (cosine similarities: 0.86 and 0.84, respectively; FIG. 13A).

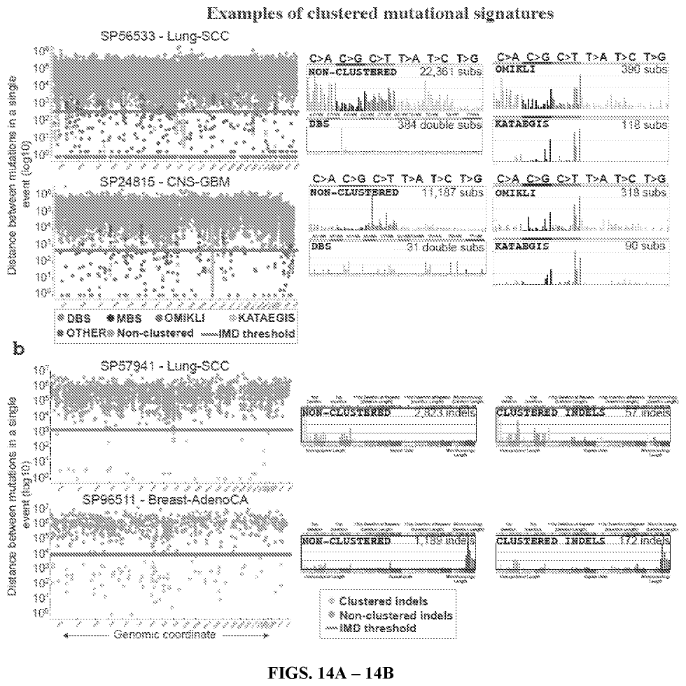

From the 9 kataegic signatures, 4 have been reported previously including 2 associated with APOBEC3 deaminases (SBS2 and SBS13) and 2 associated with canonical or non-canonical AID activities (SBS84 and SBS85; FIG. 2). SBS5 (clock-like mutagenesis) accounted for 15.0% of kataegis with most events occurring in the vicinity of AID kataegis within B-cell lymphomas. The remaining 4 kataegic signatures accounted for only 8.9% of kataegic mutations and included: SBS7a/b (ultraviolet light), SBS9 (indirect mutations from AID), and SBS37 (unknown etiology). Most kataegic signatures were strand-coordinated (FIG. 13B). Some samples exhibited consistent while others exhibited distinct signatures of clustered and non-clustered mutagenesis (FIG. 14). For example, in SP56533 (lung squamous cell carcinoma), most non-clustered and omikli substitutions were caused by tobacco signature SBS4, while kataegic events were generated by the APOBEC3 signatures (FIG. 14A). In contrast, the pattern of non-clustered substitutions in SP24815 (glioblastoma) was due to clock-like signatures SBS1 and SBS5 while omikli and kataegic events were mostly attributable to APOBEC3 (FIG. 14A).

The remaining other clustered substitutions exhibited inconsistent VAFs likely representing mutations at highly mutable genomic regions or the effects of co-occurring large mutational events such as copy number alterations (FIG. 13D).

Different cancers revealed distinct tendencies of clustered indel mutagenesis (FIG. 2). For instance, clustered indels attributed to ID3 (tobacco smoking; characterized by 1 bp deletions) were found predominately in lung cancers and significantly elevated in smokers compared to non-smokers (p-value: 0.0014; FIGS. 13C and 14B). Clustered indels due to signatures ID6 and ID8, both attributed to homologous recombination deficiency and characterized by long indels at microhomologies, were found in breast and ovarian cancers and were highly elevated in cancers with known deficiencies in homologous recombination genes (p-value: 4.9×10⁻¹¹; FIGS. 13C and 14B).

### Panorama of Clustered Driver Mutations

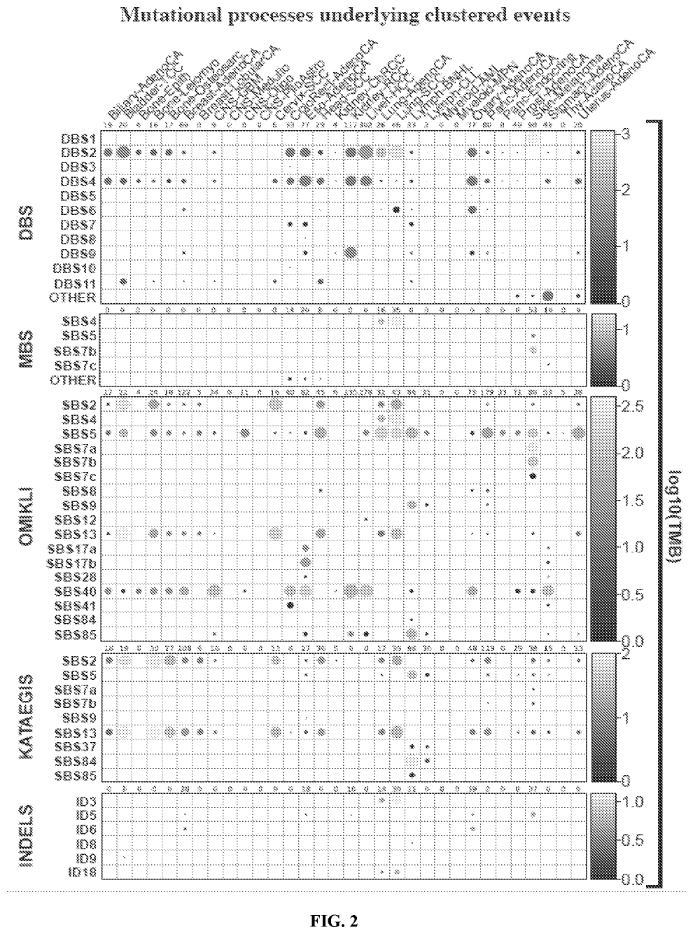

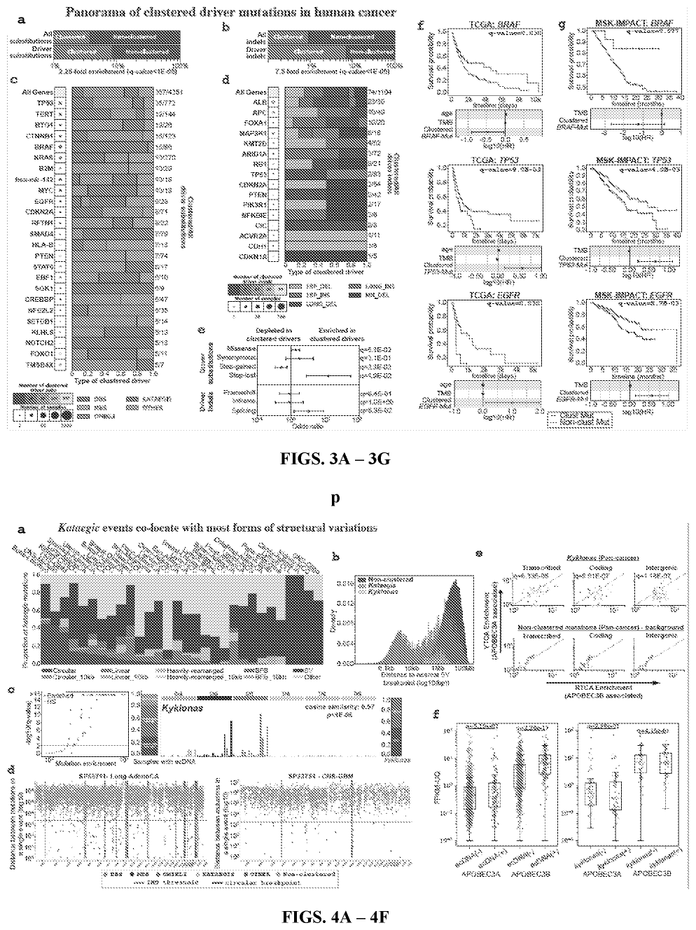

The PCAWG project elucidated a constellation of mutations putatively driving cancer development¹⁰. The disclosed data reveals significant enrichments of clustered substitutions and clustered indels amongst these driver mutations. Specifically, whereas only 3.7% of all substitutions and 0.9% of all indels are clustered events, they contribute 8.4% and 6.9% of substitution and indel drivers, respectively (q-values <1e-5; Fisher's exact tests; FIGS. 3A and 3B). Omikli accounted for 50.5% of all clustered substitution drivers, while DBSs, kataegis, and other clustered events each contributed between 14% and 18% (FIG. 3C). Clustered driver substitutions varied greatly between genes and across different cancers (FIG. 3C and FIG. 7A) with a 2.4-fold enrichment of clustered events within oncogenes compared to tumor suppressors (p-value=5.79E-03; FIGS. 7B and 7C). In some cancer genes, only a small percentage of driver events are due to clustered substitutions; examples include TP53 (4.5% clustered driver substitutions), KRAS (3.7%), and PIK3CA (2.2%). In other genes, most detected substitution drivers were clustered events; examples include: BTG1 (73.1%), SGK1 (66.6%), EBF1 (60.0%), and NOTCH2 (38.5%). Importantly, the contribution from each class of clustered events varied across driver substitutions in different genes (FIG. 3C). For instance, ultraviolet light associated DBSs comprise 93% of clustered BRAF driver events, omikli contribute 63% of clustered BTG1 driver events, and kataegis accounted for 100% of clustered NOTCH2 driver substitutions (FIG. 3C). Similar behavior was observed for clustered indel drivers with 48.7% being single-base pair indels (FIG. 3D). In some cancer genes, clustered indel drivers were rare (e.g., 2.4% of indel drivers in TP53 were clustered) whereas in others they were common (e.g., 76.6% in ALB; FIG. 3D). Clustered driver substitutions were enriched in stop-lost mutations (q-value=1.9E-02) and depleted in stop-gained mutations (q-value=3.3E-03) when compared to non-clustered drivers (FIG. 3E). Further, driver genes harboring clustered events were often differentially expressed compared to those harboring non-clustered events (FIG. 7D). For instance, clustered events within CTNNB1 and BTG1 associated with an increased expression compared to both non-clustered and wild-type expression levels for each gene (q-values <0.05). Opposite effects were observed in STAT6 and RFTNI (q-values <0.05). Collectively, these driver events were induced by the activity of multiple mutational processes including exposure to ultraviolet light, tobacco smoke, platinum chemotherapy, and AID/APOBEC3 activity; amongst others (FIG. 5E).

### Kataegic Events and Focal Amplifications

In each sample, kataegic mutations were separated into distinct events based on consistent VAFs across adjacent mutations and IMD distances greater than the sample-dependent IMD threshold. Applicant's analysis revealed that 36.2% of all kataegic events occurred within 10 kb of a structural breakpoint but not on detected focal amplifications (FIG. 4A). Additionally, 21.8% of all kataegic events occurred either on a detected focal amplification or within 10 kb of a focal amplification's structural breakpoints: 9.6% on circular extrachromosomal DNA (ecDNA), 6.3% on linear rearrangements, 3.3% within heavily rearranged events, and 2.6% associated with BFBs (FIG. 4A). Lastly, 42.0% of kataegic events were neither within 10 kb of a structural breakpoint nor on a detected focal amplification. Modelling the distribution of the distances between kataegic events and the nearest structural variations revealed a multi-modal distribution with three components (FIG. 4B): kataegis within 10 kb, ˜10 Mb, or >1.5 Mb of a detected breakpoint. Importantly, ecDNA-associated kataegis, termed kyklonas (Greek for cyclone), had ˜750 kb average distance from the nearest breakpoint with only 0.35% of kyklonic events occurring both on ecDNA and within 10 kb of a breakpoint (FIG. 4B). These results indicate that kyklonic events are not likely to have occurred due to structural rearrangements during the formation of ecDNA. In most cancer types, DBSs, MBSs, omikli, and other cluster events were not found in the vicinity of structural variations (FIGS. 15A and 15B).

Recurrent Kyklonic Mutagenesis of ecDNA

While only 9.6% of kataegic events occur within ecDNA regions, >30% of ecDNAs had one or more associated kyklonic events (FIG. 4C). The mutations within these ecDNA regions were dominated by the APOBEC3 patterns, which are characterized by strand-coordinated C>G and C>T mutations at TpCpW context and attributed to signatures SBS2 and SBS13 (p-value <1E-5; FIGS. 4C,4D, and 15C). These APOBEC3-associated events contributed 97.8% of all kyklonic events, while the remaining mutations were attributed to clock-like signature SBS5 (1.2%) and other signatures (1.0%; FIG. 15C). Further, kyklonic events exhibited an enrichment of C>T and C>G mutations at APOBEC3B-preferred RTCA compared to APOBEC3A-preferred YTCA contexts⁷ indicating that APOBEC3B likely plays an important role in the mutagenesis of circular DNA bodies (FIG. 4E). Similar levels of enrichment for RTCA contexts were also observed in both non-ecDNA kataegis and non-SV associated kataegis suggesting that APOBEC3B generally gives rise to many of the strand-coordinated kataegic events (FIG. 15D). Elevation of APOBEC3B, but not APOBEC3A, expression was observed in cancers with ecDNA compared to samples without ecDNA (3.1-fold; q-value <1E-5; FIG. 4F). Within cancers harboring ecDNA, no differences were observed in APOBEC3A/B expression between samples with and without kyklonic events (FIG. 4F).

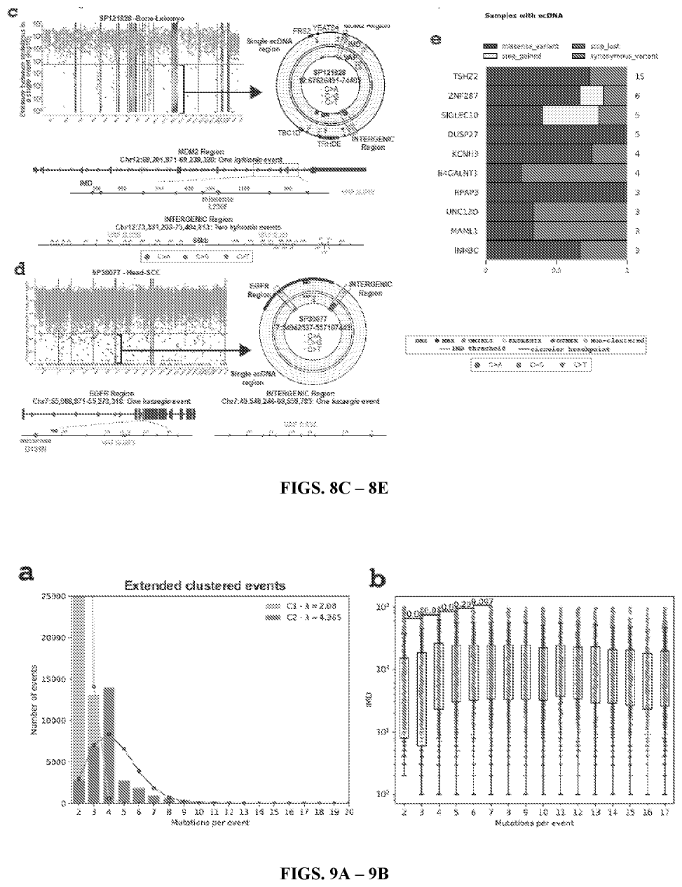

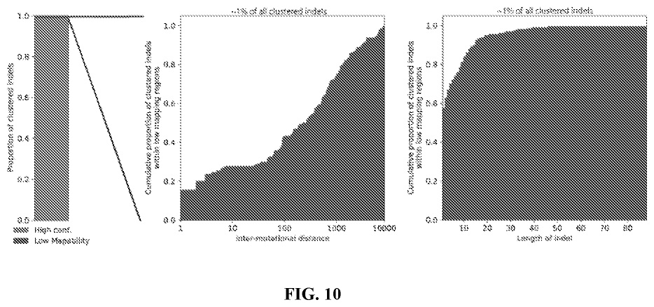

More recurrent APOBEC3 kataegis was observed across circular ecDNA regions compared to other forms of structural variations (FIG. 5A). An average of 2.5 kyklonic events were observed within ecDNA regions (range: 0 to 64 kyklonic events; 0 to 505 mutations). Recurrent kyklonas was widespread across cancer types (FIGS. 8A and 8B). For instance, glioblastomas and sarcomas exhibited an average of 5 and 86 kyklonic mutations, respectively. The average VAF of kyklonas was significantly lower than both non-ecDNA associated kataegis and all other clustered events (q-values <1e-5; FIG. 5B). Interestingly, a subset of kyklonas exhibited VAFs above 0.80 likely reflecting early mutagenesis of genomic regions that have subsequently amplified as ecDNA. Further, kyklonic events with high VAFs occurred more commonly on ecDNA harboring known cancer genes suggesting a mechanism of positive selection (FIG. 5B). Approximately 7.2% of kyklonas occurred early in the evolution of a given ecDNA population within a tumor (VAF >0.80), while the majority of kyklonic events (˜82.5%; VAF <0.5) have likely occurred after clonal amplification by recurrent APOBEC3 mutagenesis.

Recurrent kyklonic events were increased within or near known cancer-associated genes including TP53, (DK4, and MD) M2; amongst others (FIG. 5C). These recurrent kyklonas were observed across many cancers including glioblastomas, sarcomas, head and neck carcinomas, and lung adenocarcinomas (FIGS. 8C and 8D). For example, in a sarcoma sample (SP121828), 10 distinct kyklonic events overlapped a single ecDNA region with recurrent APOBEC3 activity in proximity to MIM2 resulting in a missense L230F mutation (FIG. 8C). The same ecDNA region harbored additional kyklonic events occurring within intergenic regions that have distinguishable VAF distributions implicating recurrent mutagenesis (FIG. 8C). Similarly, two distinct kyklonic events occur on an ecDNA harboring EGFR resulting in a missense mutation D191N within a head and neck cancer (FIG. 8D). Importantly, ecDNA regions with known cancer-associated genes had a significantly higher numbers of kyklonic events and mutational burdens of kyklonas compared to ecDNA regions without any known cancer-associated genes (q-values <1e-5;

FIG. 5D). Further, Applicant observed a higher co-occurrence of kyklonas with known cancer-associated genes, which were mutated 2.5 times more than ecDNA without cancer-associated genes (p-value=1.2e-5; Fisher's exact test). Overall, 41% of kyklonic events were found within the footprints of known cancer driver genes (p-value <1e-5). These enrichments cannot be accounted either by an increase in the overall mutations or by an increase in the overall clustered mutations in these samples (FIG. 5E). To understand the functional effect of kyklonas, Applicant annotated the predicted consequence of each mutation. In total, 2,247 kyklonic mutations overlapped putative cancer-associated genes, of which 4.3% occur within coding regions (FIG. 8E). Specifically, 63 resulted in missense mutations, 29 resulted in synonymous mutations, 4 introduced premature stop codons, and 1 removed a stop codon (data not shown). These downstream consequences of APOBEC3 mutagenesis suggest a contribution to the oncogenic evolution of specific ecDNA populations.

Validation of Kyklonic Events in ecDNA

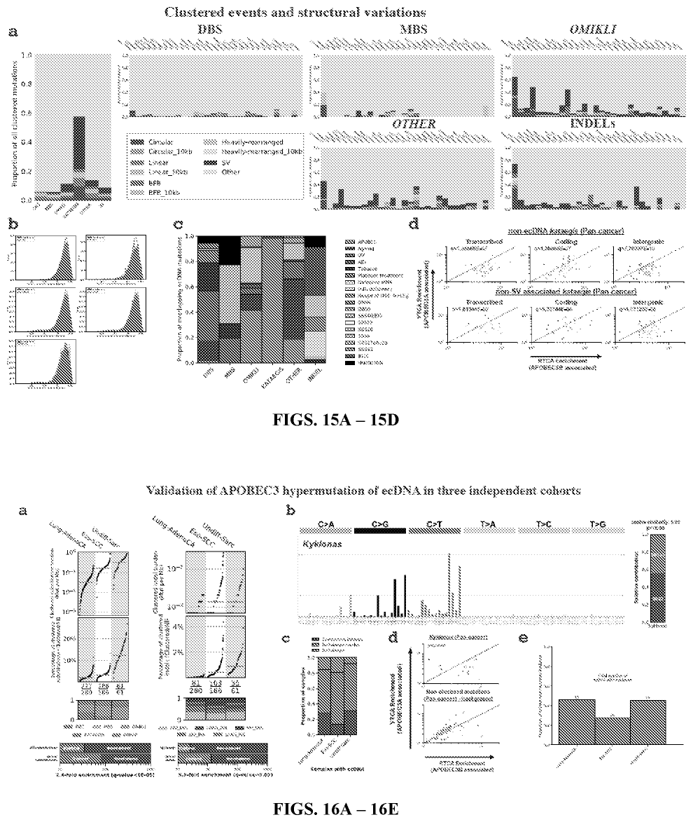

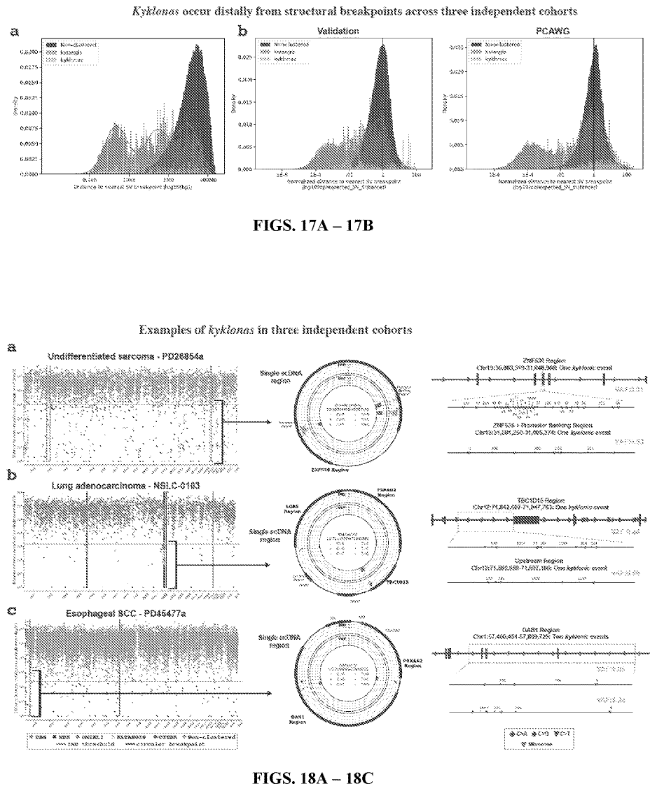

Kyklonic events were further investigated across three additional independent cohorts, including: 61 sarcomas⁴⁴, 280 lung cancers⁴⁵, and 186 esophageal squamous cell carcinomas⁴⁶. Comparable rates of clustered mutagenesis were found for both substitutions and indels as the ones reported in PCAWG with a 2.4- and 5.0-fold enrichment of clustered substitutions and indels within driver events, respectively (FIG. 16A). Across the three cohorts, 31% of samples with ecDNA exhibited kyklonas within the sarcomas, 14% within the esophageal cancers, and 28% within the lung cancers supporting the rates observed in PCAWG (FIG. 4C and FIG. 16C). Similar to the rate observed in PCAWG (36.2%), approximately 30.1% of all kataegis occurred within 10 kb of the nearest breakpoint in the validation cohort (FIG. 17A). Further, only 0.34% of kyklonic events in the validation dataset occurred closer to structural variants than expected by chance, which closely resembles the observations in the PCAWG data (0.35%; FIG. 17B). Kyklonic mutations were predominantly attributed to APOBEC3 signatures SBS2 and SBS13 (p-value <1E-05; FIG. 16B) with an enrichment of mutations at RTCA context supporting the role of APOBEC3B (FIG. 16D). A widespread recurrence of kyklonic events were observed across the sarcomas, esophageal, and lung cancers with 45%, 28%, and 46% of samples with ecDNA harboring multiple, distinct kyklonic events (FIG. 16E). An example from each cohort was selected to illustrate multiple kyklonic events occurring within single ecDNAs validating recurrent APOBEC3 hypermutation of ecDNA (FIG. 17).

### Data Sources

Somatic variant calls of single-base substitutions, small insertions and deletions, and structural variations were downloaded for the 2,583 white-listed whole-genome sequenced samples from PCAWG along with the corresponding list of consensus driver events¹⁰. Epidemiological and clinical features for all available samples were downloaded from the official PCAWG release (https://dcc.icgc.org/releases/PCAWG). The collection of whole-exome sequenced samples from TCGA along with all available clinical features were downloaded from the Genomic Data Commons (https://gdc.cancer.gov/). The MSK-IMPACT Clinical Sequencing Cohort⁴³ composed of 10,000 clinical cases was downloaded from cBioPortal (https://www.cbioportal.org/study/summary?id=msk_impact_2017). The subclassification of focal amplifications comprised of circular extrachromosomal DNA (ecDNA), linear amplifications, breakage-fusion-bridge cycles (BFBs), and heavily rearranged events, and their corresponding genomic locations were obtained for a subset of samples (n=1,291) as reported³⁴.

Experimental models used to validate clustered events were derived from previous studies using primary Hupki mouse embryonic fibroblasts (MEFs) exposed to ultraviolet light⁴¹, human induced pluripotent stem cells (iPSC) exposed to benzo[a]pyrene⁴⁰, and clonally expanded BT-474 human breast cancer cell line with episodically active APOBEC3³⁹.

Independent cohorts used to validate kyklonic events were collected from multiple sources. The 61 undifferentiated sarcomas⁴⁴ and 187 high-confidence esophageal squamous cell carcinomas⁴⁶ were downloaded from the European Genome-phenome Archive (EGAD00001004162 and EGAD00001006868, respectively). The 280 lung adenocarcinomas⁴⁵ were downloaded from dbGaP under the accession number (phs001697.v1.p1). Clustered mutations in validation samples were analyzed using the same approach as the one utilized in the original cohort.

### Detection of Clustered Events

SigProfilerSimulator (v1.0.2) was used to derive an intra-mutational distance (IMD) cutoff⁵¹ that is unlikely to occur by chance based upon the tumor mutational burden and the mutational patterns for a given sample. Specifically, each tumor sample was simulated while maintaining the sample's mutational burden on each chromosome, the +/−2 bp sequence context for each mutation, and the transcriptional strand bias ratios across all mutations. All mutations in each sample were simulated 100 times and the IMD cutoff was calculated such that 90% of the mutations below this cutoff could not appear by chance (q-value <0.01). For example, in a sample with an IMD threshold of 500 bp, one may observe 1.000 mutations within this threshold with no more than 100 mutations expected based on the simulated data (q-value <0.01). P-values were calculated using z-tests by comparing the number of real mutations and the distribution of simulated mutations that occur below the same IMD threshold. A maximum cutoff of 10 kb was used for all IMD thresholds. By generating a background distribution that reflects the random distribution of events used to reduce the false positive rate, this model also considers regional heterogeneities of mutation rates, partially attributed to replication timing and expression, and variances in clonality by correcting for mutation-rich regions and mutation-poor regions within 1 Mb windows. The 1 Mb window size has been utilized and established as an appropriate scale when considering the variability in mutation rates associated with chromatin structure, replication timing, and genome architecture^{14,52,53}. The 1 Mb window ensures that subsequent mutations likely occurred as single events using a maximum cutoff of 0.10 for differences in the variant allele frequencies (VAFs). The regional IMD cutoff was determined using a sliding window approach that calculated the fold enrichment between the real and simulated mutation densities within 1 Mb windows across the genome. The IMD cutoffs were further increased, for regions that had higher than 9-fold enrichments of clustered mutations and where >90% of the clustered mutations were found within the original data, to capture additional clustered events while maintaining the original criteria (<10% of the mutations below this cutoff appear by chance; q-value <0.01). Lastly, as VAF of mutations may confound the definition of clustered events in ecDNA, Applicant calculated the distribution of inter-event distances within recurrently mutated ecDNA while disregarding the VAF of individual mutations. This resulted in the exact same separation of kataegic events using only the inter-event distances as a criterion for the grouping of mutations into a single event.

Subsequently, all clustered mutations with consistent VAFs were classified into one of four categories (FIG. 6A). Two adjacent mutations with an IMD of 1 were classified as doublet-base substitutions. Three or more adjacent mutations each with an IMD of 1 were classified as multi-base substitutions. Two or three mutations with IMDs less than the sample-dependent threshold and with at least a single IMD greater than 1 were classified as omikli. Four or more mutations with IMDs less than the sample-dependent threshold and with at least a single IMD greater than 1 were classified as kataegis. A cutoff of four mutations for kataegis was chosen by fitting a Poisson mixture model to the number of mutations involved in a single event across all extended clustered events excluding DBSs and MBSs (data not shown). This model comprised two distributions with C1=2.08 and C2=4.37 representing omikli and kataegis, respectively. A cutoff of four mutations was used for kataegis based upon >95% contribution from the kataegis-associated distribution with events of four or more mutations. Applicant notes that there is certain ambiguity for events with 2 or 3 mutations. While the majority of these events are omikli, some of these events are likely short kataegic events (data not shown). All remaining clustered mutations with inconsistent VAFs were classified as other. Clustered indels were not classified into different classes. Applicant also performed additional quality-checks to ensure that the majority of clustered indels were mapped to high confidence regions of the genome (data not shown). Specifically, all clustered indels were aligned against a consensus list of blacklisted genomic regions developed by ENCODE⁵⁴ revealing that only 0.5% of all clustered indels overlapped regions with low mappability scores.

### Clustered Mutational Signatures Analysis

The clustered mutational catalogues of the examined samples were summarized in SBS288 and ID83 matrices using SigProfilerMatrixGenerator⁵⁵ (version 1.2.0) for each tissue type and each category of clustered events. For example, six matrices were constructed for clustered mutations found in Breast-AdenoCA: one matrix for DBSs, one matrix for MBSs, one matrix for omikli, one matrix for kataegis, one matrix for other clusters substitutions, and one matrix for clustered indels. The SBS288 classification considers the 5′ and 3′ bases immediately flanking each single-base substitution (referred to using the pyrimidine base in the Watson-Crick base pair) resulting in 96 individual mutation channels. Further, this classification considers the strand orientation for mutations that occur within genic regions resulting in three possible categories; (i) transcribed; pyrimidine base occurs on the template strand; (ii) untranscribed; pyrimidine base occurs on the coding strand; or (iii) non-transcribed; pyrimidine base occurs in an intergenic region. Note that mutations in genic regions that are bi-directionally transcribed were evenly split amongst the coding and template strand channels. Combined, this results in a classification consisting of 288 mutation channels, which were used as input for de novo signature extraction of clustered substitutions. The ID83 mutational classification has been previously described⁵⁵.

Mutational signatures were extracted from the generated matrices using SigProfilerExtractor (v1.1.0), a Python based tool that uses nonnegative matrix factorization to decipher both the number of operative processes within a given cohort and the relative activities of each process within each sample⁵⁶. The algorithm was initialized using random initialization and by applying multiplicative updates using the Kullback-Leibler divergence with 500 replicates. Each de novo extracted mutational signature was subsequently decomposed into the COSMIC (v3) set of signatures (https://cancer.sanger.ac.uk/signatures/) requiring a minimum cosine similarity of 0.80 for all reconstructed signatures. All de novo extractions and subsequent decomposition were visually inspected and, as previously done¹, manual corrections were performed for 2.2% of extractions (4 out of 180 extractions) where the total number of operative signatures was adjusted ±1. Consistent with prior visualizations¹⁰, Applicant included all cancer types within the PCAWG cohort which may comprise as few as one sample for certain cancer types. Similarly, consistent with prior visualizations¹, decomposed signature activity plots required that each cancer type have more than 2 samples and used mutation thresholds for each clustered category; 25 mutations per sample were required for doublet-base substitutions, omikli events, and other clustered mutations; 15 mutations per sample were required for multi-base substitutions and kataegic events; 10 mutations were required per sample for clustered indels.

### Experimental Validation

A subset of clustered mutational signatures was validated using previously sequenced in vitro cell line models. As done for PCAWG samples, Applicant generated a background model using SigProfilerSimulator⁵¹ to calculate the clustered IMD cutoff for each sample and partitioned each substitution into the appropriate category of clustered events. Mutational spectra were generated for each subclass within each sample using SigProfilerMatrixGenerator⁵⁵ and were compared against the de novo signatures extracted from human cancer. The cosine similarity between the in vitro mutational spectra and de novo observed clustered signatures was calculated to assess the degree of similarity. Applicant notes that the average cosine similarity between two random nonnegative vectors is 0.75, and the cosine similarities above 0.81 reflect p-values below 0.01⁵¹.

Associations with Cancer Risk Factors

Homologous recombination deficiency (HRD) was defined for breast cancers using the status of BRCA1, BRCA2, RAD51C, and PALB2⁵⁷. Samples with a germline, somatic, or epigenetic alteration in one of these genes were considered HR-deficient, while samples without any known alterations in these genes were considered HR-proficient. The number of clustered indels were compared between HR-deficient and HR-proficient samples. The smoking status of lung cancers was determined using the clinical annotation from TCGA (https://portal.gdc.cancer.gov/repository). The number of clustered indels associated with tobacco smoking (ID6) were compared between samples annotated as lifelong non-smokers and samples annotated as current and reformed smokers. The status of alcohol consumption was determined using the annotations from the official PCAWG release (https://dcc.icgc.org/releases/PCAWG). The total number of clustered indels were compared in samples annotated with no alcohol consumption and those annotated as daily and weekly drinkers.

### Expression of Driver Genes

All RNA-seq expression data was downloaded as a part of the official PCAWG release (https://dcc.icgc.org/releases/PCAWG). The relative expression data found within this release were normalized using fragments per kilobase of exon per million mapped fragment (FPKM) normalization and upper quartile normalization. The relative expressions of a gene were compared between those harboring clustered or non-clustered events. Each distribution was then normalized to the average expression of the wild-type gene. Only genes with at least 10 total events (i.e., clustered and non-clustered mutations) including at least 5 clustered events were considered for examination.

### Structural Variants and Clustered Events

The distance to the nearest structural variation breakpoint was calculated for each mutation in each subclass using the minimum distance to the nearest adjacent upstream or downstream breakpoint. Each distribution was modeled using a Gaussian mixture with an automatic selection criterion for the number of components ranging between one and five components using the minimum Bayesian information criteria (BIC) across all iterations. Modelling of kataegic events resulted in an optimal fit of three components, which was used to separate kataegic substitutions into SV-associated and non-SV associated mutations. Doublet-base substitutions and multi-base substitutions were both modelled using a single Gaussian distribution relating to non-SV associated mutations, while omikli and other clustered mutations were modelled using a mixture of two components likely reflecting leakage of smaller kataegic events contributing to a weak SV-associated distribution. To account for the frequency of breakpoints across each sample, Applicant normalized the minimum distance of each mutation to the nearest SV by calculating the expected distance between a mutation and SV for each sample using the total number of breakpoints and the overall length of a given chromosome (data not shown). After normalizing the kataegic events, Applicant observed an optimal solution of two components with one SV-associated distribution (on average each mutation occurs within one thousandth of the expected distance to nearest structural variation) and one non-SV associated distribution (on average occurring within the expected distance to the nearest structural variation). The normalized kyklonic events are consistent with the non-SV associated distribution reflecting kataegic events that occur on ecDNA typically of lengths 1-10 Mb³⁵.

### APOBEC3A/B Enrichment Analysis

The enrichment score of RTCA and YTCA penta-nucleotides quantifies the frequency for which each TpCpA>TpKpA mutation occurs at either an RTCA or YTCA context. To account for motif availability, this score is calculated using the +/−20 bp sequence context around each mutation and normalized by the number of cytosine bases and C>N mutations within the set of 41-mers surrounding each mutation of interest7.

### APOBEC3 Gene Expression and Kyklonas

All RNA-seq expression data was downloaded as a part of the official PCAWG release (https://dcc.icgc.org/releases/PCAWG). The relative expression data found within this release were normalized using fragments per kilobase of exon per million mapped fragment (FPKM) normalization and upper quartile normalization. The APOBEC3A/B normalized expression were compared between samples harboring ecDNA versus samples with no detected ecDNA and between samples with kyklonas and without kyklonas. All p-values were generated using a Mann-Whitney U test and were corrected for multiple hypothesis testing using the Benjamini-Hochberg false discovery rate procedure.

Circular ecDNA and Kataegis

The collection of ecDNA ranges were intersected with the catalog of clustered mutations, which was used to determine the overlapped mutational burden for each subclass of clustered event and the mutational spectra of overlapping kataegic events. Enrichments of events were calculated using statistical background models generated using SigProfilerSimulator⁵¹ that shuffled the dominant mutation in each clustered event across the genome (i.e., the most frequent mutation type in a single event). The decomposed kyklonic mutational spectra was generated using the decomposition module within SigProfilerExtractor⁵⁶. Only mutational signatures increasing the overall cosine similarity with at least 0.01 were used. In both the original and validation cohorts, SBS2 and SBS13 were sufficient to explain the kyklonic mutational spectra with no other known mutational signature increasing the cosine similarity with more than 0.01. Comparisons between ecDNA with and without cancer genes were performed using the set of cancer genes from the Cancer Gene Census (CGC)⁵⁸. All statistical comparisons and p-values were calculated using a two-tailed Mann-Whitney U test unless otherwise specified. For each set of tests, p-values were corrected for multiple hypothesis testing using the Benjamini-Hochberg false discovery rate procedure. The predicted effect of each overlapping variant was determined using ENSEMBL's Variant Effect Predictor tool by reporting only the most severe consequence⁵⁹.

### Overall Survival and Clustered Mutations

All survival analyses, including the generation of Kaplan-Meier curves, Cox regressions, and Log-rank tests, were performed using the Lifelines Python package (v0.24.4). Across the 30 distinct whole-genome sequenced cancer types included in the PCAWG study, only 6 cancer types contained enough samples to explore the associations between survival and overall number of clustered mutations. The sufficient sample size criteria required more than 50 samples with survival endpoints with at least 30 of the samples with an observed clustered event. Each cancer type was analyzed separately by comparing the survival of samples with a high clustered mutational burden (top 80^{th} percentile across a given cancer type) to the survival of samples with a low clustered mutational burden (bottom 20^{th} percentile across a given cancer type).

Analysis of whole-exome sequenced samples from TCGA was altered to reflect the limited resolution for identifying clustered mutations within the exome. Specifically, SigProfilerSimulator (v1.0.2)⁵¹ was used to derive an IMD cutoff for each sample based on the tumor mutational burden within the exome and the mutational patterns for a given sample. Mutations were randomly shuffled while maintaining the mutational burden within the exome of each chromosome, the +/−2 bp sequence context for each mutation, and the transcriptional strand bias ratios across all mutations. Each sample was simulated 100 times and an IMD cutoff was calculated using the same methods as outlined for the detection of clustered events within PCAWG. Due to the limited number of detected events, 22 cancer types had sufficient data to perform survival analysis. Each cancer type was analyzed separately by comparing samples with at least a single clustered event to samples with no detected clustered events within the exome.

For both PCAWG and TCGA analyses, survival distributions within a given cancer type were compared using a Log-rank test. Cox regressions were performed to determine hazards ratios and to correct for age and total mutational burden. All p-values were also corrected for multiple hypothesis testing using the Benjamini-Hochberg false discovery rate procedure.

To investigate differential survival associated with the detection of clustered events within cancer driver genes, Kaplan-Meier survival curves were compared between individuals harboring clustered versus non-clustered mutations within a given cancer driver gene. The distributions were compared using a Log-rank test. Cox regressions were performed to determine the hazards ratios and to correct for age, total mutational burden, and cancer type across TCGA. Cox regressions performed for the MSK-IMPACT cohort were corrected for total mutational burden and cancer type. No corrections were performed for age as these metadata were not available for the MSK-IMPACT cohort. All p-values were also corrected for multiple hypothesis testing using the Benjamini-Hochberg false discovery rate procedure.

### Validation of Kyklonas in Three Cohorts

All three validation cohorts were analyzed analogous to the PCAWG cohorts. Specifically, clustered mutations were classified by calculating a sample-dependent IMD threshold for clustered versus non-clustered mutations using a background model generated by SigProfilerSimulator⁵¹. All clustered mutations were subclassified into either DBS, MBS, omikli, kataegis, or other mutations. AmpliconArchitect (version 1.2) was used to determine regions of focal amplifications⁶⁰, which were utilized for subsequent validation of kyklonic events by overlapping kataegic events with all detected focal amplifications. The decomposed kyklonic mutational spectra was generated using the decomposition module within SigProfilerExtractor⁵⁶. Only mutational signatures increasing the overall cosine similarity with at least 0.01 were used. In both the original and validation cohorts, SBS2 and SBS13 were sufficient to explain the kyklonic mutational spectra with no other known mutational signature increasing the cosine similarity with more than 0.01.

### Data Availability

No data were generated specifically for this study. All data were and can be downloaded from the appropriate links, repositories, and references. Specifically, for the discovery cohort, all data and metadata were obtained from the official PCAWG release: https://dcc.icgc.org/releases/PCAWG. All data and metadata for TCGA samples were obtained from GDC: https://gdc.cancer.gov/. Genomics data for clonally expanded cell lines were downloaded from European Genome-phenome Archive: EGAD00001004201, EGAD00001004203, and EGAD00001004583. For the three validation cohorts, datasets were downloaded as submitted by the original publications and genomics data were downloaded from their respective repositories: EGAD00001004162 for 61 undifferentiated sarcomas⁴⁴ (European Genome-phenome Archive), EGAD00001006868 for 187 high-confidence esophageal squamous cell carcinomas⁴⁶ (European Genome-phenome Archive), and phs001697.v1.p1 for 280 lung adenocarcinomas⁴⁵ (dbGaP). Somatic mutations and metadata for the MSK-IMPACT Clinical Sequencing Cohort composed of 10,000 clinical cases⁴² were downloaded from cBioPortal: https://www.cbioportal.org/study/summary?id=msk_impact_2017.

### Code Availability

The SigProfiler compendium of tools are developed as Python packages and are freely available for installation through PyPI or directly through GitHub (https://github.com/AlexandrovLab/). For all tools, each package is fully functional, free, and open sourced distributed under the permissive 2-Clause BSD License and are accompanied by extensive documentation: (i) SigProfilerMatrixGenerator⁵⁵ (version 1.2.0; https://github.com/AlexandrovLab/SigProfilerMatrixGenerator); (ii) SigProfilerSimulator⁵¹: (version 1.0.2; https://github.com/AlexandrovLab/SigProfilerSimulator); (iii) SigProfilerExtractor⁵⁶: (version 1.1.0; https://github.com/AlexandrovLab/SigProfilerExtractor). Each SigProfiler tool also has an R wrapper available for installation through the GitHub repositories. AmpliconArchitect³⁴ (version 1.2) is also freely available and can downloaded from https://github.com/virajbdeshpande/AmpliconArchitect. The core computational pipelines used by the PCAWG Consortium for alignment, quality control and variant calling are available to the public at https://dockstore.org/search?search=pcawg under the GNU General Public License v.3.0, which allows for reuse and distribution.

### Discussion

Clustered mutagenesis in cancer can occur through different mutational processes, with AID/APOBEC3 deaminases playing the most prominent role. In addition to enzymatic deamination, other endogenous and exogenous sources imprint many of the observed clustered indels and substitutions. Importantly, a multitude of mutational processes can give rise to omikli events including tobacco carcinogens and exposure to ultraviolet light. Clustered substitutions and indels were highly enriched in driver events and associated with differential gene expression, implicating them in cancer development and cancer evolution. Some clustered mutational signatures are associated with known cancer risk factors or the activity or failure of DNA repair processes. Importantly, clustered mutations in TP53, EGFR, and BRAF associated with changes in overall survival and can be detected in most types of sequencing data, including clinically actionable targeted panels such as MSK-IMPACT. Clustered mutations with clinical significance were also detected in KIT, KMT2C, ELF3, APC and AIIDIA.

A large proportion of kataegic events occur within 10 kb of detected structural variant breakpoints with a mutational pattern suggesting the activity of APOBEC3. Multiple distinct kataegic events, independent of detected breakpoints, were observed on circular ecDNA, termed kyklonas, implicating recurrent APOBEC3 mutagenesis. The circular topology of ecDNAs⁴⁷ and their rapid replication patterns are reminiscent of the structure and behavior of the circular genomes of several double stranded DNA based pathogens including herpesviruses, papillomaviruses, and polyomaviruses³²⁻³⁵. Importantly, prior pan-virome studies have shown that these double stranded DNA viral genomes often manifest mutations from APOBEC3 enzymes⁴⁸⁻⁵⁰. As such, recurrent APOBEC3 mutagenesis on ecDNA is likely representative of an anti-viral response where the ecDNA viral-like structure is treated as an infectious agent and attacked by APOBEC3 enzymes. ecDNAs harbor a plethora of cancer-associated genes and are responsible for many gene amplification events that can accelerate tumor evolution. Repeated mutagenic attacks of these ecDNA reveals functional effects within known oncogenes implicating additional modes of oncogenesis that may ultimately contribute to subclonal tumor evolution, subsequent evasion to therapy, and clinical outcome. Further investigations with large-scale clinically annotated whole-genome sequenced cancers are required to fully understand the clinical implications of clustered mutations and kyklonas.

### SUMMARY

The clinical utility of detecting clustered events in driver genes was evaluated by comparing the survival amongst individuals with clustered mutations versus individuals harboring non-clustered mutations within each driver gene across all whole-exome sequenced samples in TCGA. For each of these comparisons, Applicant performed Cox regressions considering the effects from age and TMB while correcting for cancer type and multiple hypothesis testing. These results were validated in targeted panel sequencing data from the Memorial Sloan Kettering-Integrated Mutation Profiling of Actionable Cancer Targets (MSK-IMPACT) cohort^{42,43}. These analyses revealed a significant difference in survival between individuals with clustered and individuals with non-clustered mutations detected in TP53, EGFR, and BRAF. Specifically, individuals with clustered events within BRAF had a better overall survival compared to ones with non-clustered events (q-values <0.05; FIGS. 7F and 7G). Conversely, in both TCGA and MSK-IMPACT, individuals with clustered mutations in TP53 or EGFR exhibited a significantly worse outcome compared to ones with non-clustered mutations in each of these genes (q-values <0.05; FIGS. 3F and 3G).

### Experiment No. 2:

### Determining the Number of Clustered Mutations in Omikli and Kataegic Events.

To determine the cutoff of the number of mutations in an omikli versus a kataegic event, Applicant modelled the distribution of clustered event sizes (excluding DBSs, MBSs, and other clustered events with disagreeable variant allele frequencies) using a mixture of two Poisson distributions (FIG. 9A). The modelling also excluded clustered mutations from skin melanomas, that contribute a disproportionate number of DBS events, and clustered mutations from lymphomas, that contribute a large proportion of canonical and non-canonical AID kataegis. The first component, corresponding with omikli events (gold), had an average of 2.1 mutations per event, while the second component, corresponding to larger kataegic events (teal), had an average of 4.4 mutations per events. Using the posterior probabilities of each distribution, Applicant calculated the likelihood of a given clustered event belonging to a specific component. Events comprised of four or more mutations were attributed to the kataegic component with >95% probability. Further, Applicant assessed the IMD distributions of different sized events revealing approximately a 2-fold increase in average IMD between events possessing 3 and 4 mutations supporting the activity of two separate mutational processes (FIG. 9B).

Analyzing the mapping scores of clustered indels. Applicant examined the mapping scores across the genome for clustered indels to ensure that the majority of events fall within high confidence regions. For this analysis Applicant used a consensus list of blacklisted genomic regions developed by ENCODE1 and the complete set of clustered indels as identified from the 2,583 PCAWG samples.

### Experiment No. 3

Using the MSK-MET targeted panel sequencing cohort, differential survival was observed across four genes in primary diseases including EGFR in non-small cell lung cancers, KIT in gastrointestinal stromal tumors, KMT2 C in bladder cancers, and ELF3 in bladder cancers and across two genes in metastatic diseases including AP (in colorectal cancers and ARID1A in bladder cancers. All associations resulted in a worse overall outcome with the presence of a clustered mutation within the gene of interest. See FIGS. 11A and 11B.

### EQUIVALENTS

Thus, it should be understood that although the present disclosure has been specifically disclosed by preferred embodiments and optional features, modification, improvement and variation of the disclosure embodied therein herein disclosed can be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. Several references are identified by an Arabic number, and the full bibliographic citation or these references are provided below. In case of conflict, the present specification, including definitions, will control.

### REFERENCES

1 Alexandrov, L. B. et al. The repertoire of mutational signatures in human cancer. Nature 578, 94-101, doi: 10.1038/s41586-020-1943-3 (2020).
2 Matsuda, T., Kawanishi, M., Yagi, T., Matsui, S. & Takebe, H. Specific tandem GG to TT base substitutions induced by acetaldehyde are due to intra-strand crosslinks between adjacent guanine bases. Nucleic Acids Res 26, 1769-1774, doi: 10.1093/nar/26.7.1769 (1998).
3 Nik-Zainal, S. et al. Mutational processes molding the genomes of 21 breast cancers. Cell 149, 979-993, doi: 10.1016/j.cell.2012.04.024 (2012).
4 de Gruijl, F. R., van Kranen, H. J. & Mullenders, L. H. UV-induced DNA damage, repair, mutations and oncogenic pathways in skin cancer. J Photochem Photobiol B 63, 19-27 (2001).
5 Brash, D. E. UV signature mutations. Photochem Photobiol 91, 15-26, doi: 10.1111/php.12377 (2015).
6 Mas-Ponte, D. & Supek, F. DNA mismatch repair promotes APOBEC3-mediated diffuse hypermutation in human cancers. Nat Genet 52, 958-968, doi: 10.1038/s41588-020-0674-6 (2020).
7 Chan, K. et al. An APOBEC3A hypermutation signature is distinguishable from the signature of background mutagenesis by APOBEC3B in human cancers. Nat Genet 47, 1067-1072, doi: 10.1038/ng.3378 (2015).
8 Taylor, B. J. et al. DNA deaminases induce break-associated mutation showers with implication of APOBEC3B and 3A in breast cancer kataegis. Elife 2, e00534, doi: 10.7554/eLife.00534 (2013).
9 Boichard, A., Tsigelny, I. F. & Kurzrock, R. High expression of PD-1 ligands is associated with kataegis mutational signature and APOBEC3 alterations. Oncoimmunology 6, e1284719, doi: 10.1080/2162402X.2017.1284719 (2017).
10 Consortium, I. T. P.-C. A. o. W. G. Pan-cancer analysis of whole genomes. Nature 578, 82-93, doi: 10.1038/s41586-020-1969-6 (2020).
11 Alexandrov, L. B. et al. Signatures of mutational processes in human cancer. Nature 500, 415-421, doi: 10.1038/nature12477 (2013).
12 Lawrence, M. S. et al. Mutational heterogeneity in cancer and the search for new cancer-associated genes. Nature 499, 214-218, doi: 10.1038/nature12213 (2013).
13 Supek, F. & Lehner, B. Clustered Mutation Signatures Reveal that Error-Prone DNA Repair Targets Mutations to Active Genes. Cell 170, 534-547 e523, doi: 10.1016/j.cell.2017.07.003 (2017).
14 Buisson, R. et al. Passenger hotspot mutations in cancer driven by APOBEC3A and mesoscale genomic features. Science 364, doi: 10.1126/science.aaw2872 (2019).
15 Greenman, C., Wooster, R., Futreal, P. A., Stratton, M. R. & Easton, D. F. Statistical analysis of pathogenicity of somatic mutations in cancer. Genetics 173, 2187-2198, doi: 10.1534/genetics.105.044677 (2006).
16 Martincorena, I. et al. Universal Patterns of Selection in Cancer and Somatic Tissues. Cell 171, 1029-1041 e1021, doi: 10.1016/j.cell.2017.09.042 (2017).
17 Hainaut, P. & Pfeifer, G. P. Patterns of p53 G->T transversions in lung cancers reflect the primary mutagenic signature of DNA-damage by tobacco smoke. Carcinogenesis 22, 367-374 (2001).
18 Pfeifer, G. P., You, Y. H. & Besaratinia, A. Mutations induced by ultraviolet light. Mutat Res 571, 19-31, doi: 10.1016/j.mrfmmm.2004.06.057 (2005).
19 Roberts, S. A. et al. Clustered mutations in yeast and in human cancers can arise from damaged long single-strand DNA regions. Mol Cell 46, 424-435, doi: 10.1016/j.molcel.2012.03.030 (2012).
20 Kasar, S. et al. Whole-genome sequencing reveals activation-induced cytidine deaminase signatures during indolent chronic lymphocytic leukaemia evolution. Nat Commun 6, 8866, doi: 10.1038/ncomms9866 (2015).
21 Roberts, S. A. et al. An APOBEC cytidine deaminase mutagenesis pattern is widespread in human cancers. Nat Genet 45, 970-976, doi: 10.1038/ng.2702 (2013).
22 Burns, M. B., Temiz, N. A. & Harris, R. S. Evidence for APOBEC3B mutagenesis in multiple human cancers. Nat Genet 45, 977-983, doi: 10.1038/ng.2701 (2013).
23 Petljak, M. et al. The APOBEC3A deaminase drives episodic mutagenesis in cancer cells. bioRxiv, 2021.2002.2014.431145, doi: 10.1101/2021.02.14.431145 (2021).
24 Bogerd, H. P., Wiegand, H. L., Doehle, B. P., Lueders, K. K. & Cullen, B. R. APOBEC3A and APOBEC3B are potent inhibitors of LTR-retrotransposon function in human cells. Nucleic Acids Res 34, 89-95, doi: 10.1093/nar/gkj416 (2006).
25 Malim, M. H. & Bieniasz, P. D. HIV Restriction Factors and Mechanisms of Evasion. Cold Spring Harb Perspect Med 2, a006940, doi: 10.1101/cshperspect.a006940 (2012).
26 Malim, M. H. Natural resistance to HIV infection: The Vif-APOBEC interaction. C R Biol 329, 871-875, doi: 10.1016/j.crvi.2006.01.012 (2006).
27 Venkatesan, S. et al. Perspective: APOBEC mutagenesis in drug resistance and immune escape in HIV and cancer evolution. Ann Oncol 29, 563-572, doi: 10.1093/annonc/mdy003 (2018).
28 Chen, H. et al. APOBEC3A is a potent inhibitor of adeno-associated virus and retrotransposons. Curr Biol 16, 480-485, doi: 10.1016/j.cub.2006.01.031 (2006).
29 Harris, R. S. & Dudley, J. P. APOBECs and virus restriction. Virology 479-480, 131-145, doi: 10.1016/j.virol.2015.03.012 (2015).
30 Harris, R. S. et al. DNA deamination mediates innate immunity to retroviral infection. Cell 113, 803-809, doi: 10.1016/s0092-8674 (03) 00423-9 (2003).
31 Maciejowski, J. et al. APOBEC3-dependent kataegis and TREX1-driven chromothripsis during telomere crisis. Nat Genet 52, 884-890, doi: 10.1038/s41588-020-0667-5 (2020).
32 Turner, K. M. et al. Extrachromosomal oncogene amplification drives tumour evolution and genetic heterogeneity. Nature 543, 122-125, doi: 10.1038/nature21356 (2017).
33 Koche, R. P. et al. Extrachromosomal circular DNA drives oncogenic genome remodeling in neuroblastoma. Nat Genet 52, 29-34, doi: 10.1038/s41588-019-0547-z (2020).
34 Kim, H. et al. Extrachromosomal DNA is associated with oncogene amplification and poor outcome across multiple cancers. Nat Genet 52, 891-897, doi: 10.1038/s41588-020-0678-2 (2020).
35 Verhaak, R. G. W., Bafna, V. & Mischel, P. S. Extrachromosomal oncogene amplification in tumour pathogenesis and evolution. Nat Rev Cancer 19, 283-288, doi: 10.1038/s41568-019-0128-6 (2019).
36 Cancer Genome Atlas Research, N. et al. The Cancer Genome Atlas Pan-Cancer analysis project. Nat Genet 45, 1113-1120, doi: 10.1038/ng.2764 (2013).
37 Green, A. M. et al. APOBEC3A damages the cellular genome during DNA replication. Cell Cycle 15, 998-1008, doi: 10.1080/15384101.2016.1152426 (2016).
38 Stenglein, M. D., Burns, M. B., Li, M., Lengyel, J. & Harris, R. S. APOBEC3 proteins mediate the clearance of foreign DNA from human cells. Nat Struct Mol Biol 17, 222-229, doi: 10.1038/nsmb.1744 (2010).
39 Petljak, M. et al. Characterizing Mutational Signatures in Human Cancer Cell Lines Reveals Episodic APOBEC Mutagenesis. Cell 176, 1282-1294 e1220, doi: 10.1016/j.cell.2019.02.012 (2019).
40 Kucab, J. E. et al. A Compendium of Mutational Signatures of Environmental Agents. Cell 177, 821-836 e816, doi: 10.1016/j.cell.2019.03.001 (2019).
41 Liu, Z. et al. Human tumor p53 mutations are selected for in mouse embryonic fibroblasts harboring a humanized p53 gene. Proc Natl Acad Sci USA 101, 2963-2968, doi: 10.1073/pnas.0308607101 (2004).
42 Cheng, D. T. et al. Memorial Sloan Kettering-Integrated Mutation Profiling of Actionable Cancer Targets (MSK-IMPACT): A Hybridization Capture-Based Next-Generation Sequencing Clinical Assay for Solid Tumor Molecular Oncology. J Mol Diagn 17, 251-264, doi: 10.1016/j.jmoldx.2014.12.006 (2015).
43 Zehir, A. et al. Mutational landscape of metastatic cancer revealed from prospective clinical sequencing of 10,000 patients. Nat Med 23, 703-713, doi: 10.1038/nm.4333 (2017).
44 Steele, C. D. et al. Undifferentiated Sarcomas Develop through Distinct Evolutionary Pathways. Cancer Cell 35, 441-456 e448, doi: 10.1016/j.ccell.2019.02.002 (2019).
45 Zhang, T. et al. Genomic and evolutionary classification of lung cancer in never smokers. Nat Genet 53, 1348-1359, doi: 10.1038/s41588-021-00920-0 (2021).
46 Moody, S. et al. Mutational signatures in esophageal squamous cell carcinoma from eight countries of varying incidence. medRxiv, 2021.2004.2029.21255920, doi: 10.1101/2021.04.29.21255920 (2021).
47 Wu, S. et al. Circular ecDNA promotes accessible chromatin and high oncogene expression. Nature 575, 699-703, doi: 10.1038/s41586-019-1763-5 (2019).
48 Cheng, A. Z. et al. Epstein-Barr virus BORF2 inhibits cellular APOBEC3B to preserve viral genome integrity. Nat Microbiol 4, 78-88, doi: 10.1038/s41564-018-0284-6 (2019).
49 Poulain, F., Lejeune, N., Willemart, K. & Gillet, N. A. Footprint of the host restriction factors APOBEC3 on the genome of human viruses. PLOS Pathog 16, e1008718, doi: 10.1371/journal.ppat.1008718 (2020).
50 Zhu, B. et al. Mutations in the HPV16 genome induced by APOBEC3 are associated with viral clearance. Nat Commun 11, 886, doi: 10.1038/s41467-020-14730-1 (2020).

1. A method of treating inhibiting the growth of a cancer cell or treating a cancer in a subject in need thereof, wherein the subject has a clustered mutation in one or more of TP53, EGFR, KIT, KMT2C, ELF3, APC and ARID1A or lacks a clustered mutation in a BRAF gene in a sample isolated from the subject, comprising administering an aggressive therapy to the subject, thereby inhibiting the growth of the cancer cell or treating the cancer in the subject.

2. The method of claim 1, wherein the cancer cell or cancer is selected from a carcinoma, a sarcoma, or a blood cancer.

3-10. (canceled)

11. The method of claim 1, wherein the subject has clustered mutations in two or more of TP53, EGFR, KIT, KMT2C, ELF3, APC and ARID1A.

12. The method of claim 1, wherein the subject further lacks a clustered mutation in BRAF.

13. (canceled)

14. A method for selecting a cancer patient for an aggressive therapy comprising detecting at least one clustered mutation in a gene selected from one or more of TP53, EGFR, KIT, KMT2C, ELF3, APC and ARID1A or lacks a clustered mutation in a BRAF gene in a sample isolated from the subject wherein the subject is selected for the therapy if the clustered mutation of one or more of TP53, EGFR, KIT, KMT2C, ELF3, APC and ARID1A is detected in the sample isolated from the cancer patient or the clustered mutation in the BRAF gene is not detected in the sample.

15. The method of claim 14, wherein the cancer cell or cancer is selected from a carcinoma, a sarcoma or a blood cancer.

16-23. (canceled)

24. The method of claim 14, wherein the subject has clustered mutations in two or more of TP53, EGFR, KIT, KMT2C, ELF3, APC and ARID1A.

25. The method of claim 14, wherein the subject further lacks a clustered mutation in BRAF.

26. (canceled)

27. A method for identifying whether a cancer patient is likely to experience a relatively longer or shorter overall survival comprising assaying for at least one clustered mutation in a gene selected from one or more of TP53, EGFR, KIT, KMT2C, ELF3, APC, ARID1A or in a sample isolated from the patient, wherein the patient is likely to experience longer overall survival if the clustered mutation is detected in BRAF and the patient is likely to experience shorter overall survival if the clustered mutation is detected in one or more of TP53, EGFR, KIT, KMT2C, ELF3, APC and ARID1A.

28. The method of claim 27, further comprising administering an aggressive chemotherapy or an aggressive immunotherapy to the patient having the clustered mutation in the one or more of TP53, EGFR, KIT, KMT2C, ELF3, APC and ARID1A or lacks a clustered mutation in a BRAF gene.

29. The method of claim 27, wherein the cancer cell or cancer is selected from a carcinoma, a sarcoma or a blood cancer.

30-37. (canceled)

38. The method of claim 27, wherein the subject has two or more clustered mutations in TP53, EGFR, KIT, KMT2C, ELF3, APC and ARID1A or lacks a clustered mutation in a BRAF gene.

39. The method of claim 27, wherein the subject further lacks a clustered mutation in BRAF.

40. (canceled)
